(19) United States
(12) Patent Application Publication
(10) Pub. No.: US 2022/0378696 A1
(43) Pub. Date: Dec. 1, 2022

(54) TARGET DELIVERY OF NON-BIOLOGICS THROUGH NANOTECHNOLOGY FOR TISSUE REPAIR

(71) Applicant: BOARD OF REGENTS, THE UNIVERSITY OF TEXAS SYSTEM, Austin, TX (US)

(72) Inventors: Michael CHO, Arlington, TX (US); Edidiong Inwang Inyang, Arlington, TX (US)

(21) Appl. No.: 17/769,892

(22) PCT Filed: Oct. 16, 2020
(86) PCT No.: PCT/US2020/056065

§ 371 Date: Apr. 18, 2022

Related U.S. Application Data
(60) Provisional application No. 62/915,986, filed on Oct. 16, 2019.

(51) Int. Cl. A61K 9/00; A61K 9/51; A61K 38/17; A61K 31/198; A61K 31/765; A61P 25/00; A61K 47/69; A61K 47/62; A61K 47/60; A61K 47/54

(52) U.S. Cl. CPC A61K 9/0085; A61K 9/5153; A61K 38/178; A61K 31/198; A61K 31/765; A61P 25/00; A61K 47/6937; A61K 47/62; A61K 47/60; A61K 47/542

(57) ABSTRACT

Provided herein are compositions and methods for diagnosis and therapy through targeted nano-delivery to injured brain endothelium. In some aspects, the compositions comprise a population of polyester derived nanoparticles, wherein each polyester derived nanoparticle comprises a) a therapeutic agent encapsulated therein for treating traumatically injured, inflamed, diseased, or disrupted endothelial cells, and b) a targeting ligand bound to the nanoparticle, wherein the targeting ligand binds to a biomarker for the injured, inflamed, diseased, or disrupted endothelial cells, are provided. The nanoparticles can be used for targeting difficult-to-reach injury sites, including the blood brain barrier and brain tissue.

### CROSS-REFERENCE TO RELATED APPLICATIONS

This application claims the benefit of U.S. Provisional Application No. 62/915,986 filed Oct. 16, 2019 which is hereby incorporated in its entirety and for all purposes.

### FIELD

This disclosure relates generally to the delivery of therapeutics through nanotechnology.

### BACKGROUND

Traumatic brain injury (TBI) is one of the major causes of emergency visits and hospitalization. In 2010, the Centers for Disease Control reported about 2.5 million emergency department visits, hospitalizations, and deaths here in the United States alone. TBIs are also caused by an explosive blast or blunt force to the head especially among those who serve in the U.S. military. The trauma can lead to endothelial cell detachment, tight junction disruption, and altered blood-brain barrier (BBB) permeability. One of the unique features of BBB is the regulation of biotransport through a monolayer of brain endothelial cells (BECs). BECs are highly regulated in their structure and function by the tight junction complex that is composed of, among many molecules, zonula occludens (ZO-1) and the occludins family. Only particles with a molecular mass of less than 500 Daltons can cross the BBB efficiently. However, the structural integrity of the BBB can be mechanically and biochemically compromised, allowing harmful substances to extravasate into the brain. The leaky brain endothelium, in turn, may lead to secondary brain injury.

Although brain trauma is increasingly better understood, it nonetheless remains elusive whether reparative treatments are plausible. This is rather important because a recent study suggests that approximately 320,000 soldiers may have experienced mild TBI during the Iraq and Afghanistan wars and that such injuries most often lead to cognitive degeneration and post-traumatic stress disorder. However, there are only a limited number of therapeutic treatments currently available, and in most cases, they are confined to identification and treatment of only the symptoms. Pharmacological selective serotonin reuptake inhibitors, for example, have been approved by FDA, and some non-pharmacological treatments such as cognitive behavioral therapy may also be effective.

There is a need for compositions and methods that specifically target (diagnostic) and repair (therapeutic) the injured brain endothelium, or stated in another way, there is a need for the development of theragnostic treatment. The compositions, systems, and methods disclosed herein address these and other needs.

### SUMMARY

Provided herein are compositions and methods for diagnosis and therapy through targeted nano-delivery to injured brain endothelium. The compositions can be derived from engineered polymeric nanoparticles that encapsulate therapeutic non-biologics and decorated with ligands to specifically target injured endothelium. The properties of the polymeric nanoparticles can be tunable, thereby facilitating a targeted drug delivery strategy. The strategy described herein can be used to provide a theragnostic approach for treatment of modulated brain endothelium associated with TBI.

In some aspects, compositions comprising a population of polyester derived nanoparticles, wherein each polyester derived nanoparticle comprises a) a therapeutic agent encapsulated therein for treating traumatically injured, inflamed, diseased, or disrupted endothelial cells, and b) a targeting ligand bound to the nanoparticle, wherein the targeting ligand binds to a biomarker for the injured, inflamed, diseased, or disrupted endothelial cells, are provided.

The population of nanoparticles can have an average particle size ranging from about 10 nm to about 1000 nm, such as from about 100 nm to about 500 nm, or from about 120 nm to about 300 nm. The polydispersity of the population of nanoparticles is preferably 0.1 or less. The polyester derived nanoparticles may comprise polylactic acid (PLA), poly glycolic acid, poly (lactic-co-glycolic acid; PLGA) or combinations thereof, preferably the nanoparticles comprise poly (lactic-co-glycolic acid). In some examples, the weight average molecular weight of the polyester derived nanoparticles can be 5,000 to 100,000, from 10,000 to 50,000, or from 24,000 to 30,000.

In some aspect of the nanoparticle compositions disclosed herein, the therapeutic agent when administered alone, exhibits negligible to no oral bioavailability. Many therapeutic agents are not absorbed by the gut, and thus the current delivery method relies on intravenous injection or otherwise. Encapsulating the therapeutic agents in the biocompatible polyester derived nanoparticles, the therapeutic agents can be delivered to an injury site, for example in the brain, through orally administering the nanoparticles. Accordingly, the present disclosure provides delivery methods of therapeutics for difficult-to-reach injury sites, including the blood brain barrier and brain tissue.

The therapeutic agent encapsulated in the nanoparticle can be a non-biologic material. For example, the therapeutic agent can comprise a surfactant, such as a poloxamer surfactant that has been demonstrated to seal the membrane of damaged cells and repair cellular function. Poloxamer 188 (P188) has been shown to reconstitute the viability and functionality of astrocytes by restoring the calcium dynamics and minimizing oxidative stress. The therapeutic agent may further comprise an antioxidant, doxycycline, or a combination thereof that are used in the treatment of traumatic injuries. In some examples, the therapeutic agent comprises an antioxidant such as N-acetylcysteine (NAC). Indeed, P188 is not absorbed by the gut when administered alone and the current delivery method relies on intravenous injection. Encapsulating multiple agents (e.g., P188 and antioxidant, and optionally one or more additional therapeutic agents) in biocompatible nanoparticles, the therapeutic agents can be delivered to the injury site in the brain through orally administering the nanoparticles.

The therapeutic agent and nanoparticles can be present in a weight ratio from 50:1 to 500:1, from 100:1 to 500:1, or from 150:1 to 400:1.

As disclosed herein, the nanoparticles can be used for targeting a traumatic injury, such as traumatic brain injury. In these examples, the nanoparticle comprises a targeting ligand that binds to a biomarker for the traumatic injury. Such biomarkers can be selected from P-selectin, E-selectin, or L-selectin. The targeting ligand can be in the form of an antibody, a small molecule, a peptide, a carbohydrate, an siRNA, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof. In some examples, the targeting ligand comprises P-selectin glycoprotein ligand-1 (PSGL-1).

Methods for treating traumatic injury in a subject in need thereof are also disclosed. The method can include administering to the subject a therapeutically effective amount of a nanoparticle composition disclosed herein. The composition may exhibit a burst release followed by sustained release of one or more of the therapeutic agents. For example, the when the therapeutic agents include a surfactant and an antioxidant, the composition may exhibit an immediate burst release followed by a sustained release of the surfactant; and an immediate burst release of the antioxidant. The composition can be administered by any suitable route, preferably intranasally or orally.

The subject in need of treatment can be diagnosed with an acute traumatic brain injury or a chronic traumatic brain injury. In some examples, the composition repairs leaky endothelium. In some embodiments, the compositions may also be used to treat diabetes and neuropathy. Indeed, glucose transporter1 (GLUT1) which is a major glucose transporter at the blood-brain barrier are colocalized with tight junction structures that regulate diffusion across the blood brain barrier (BBB). Neurons derive most of their energy from the metabolism of glucose transported into the brain from the blood. Traumatic brain injury (TBI) and addiction to psychostimulant drugs (PSDs, e.g., cocaine) or alcohol are known to cause toxicity due to oxidative and may compromise the integrity of the blood-brain barrier (BBB). The permeability of glucose in a brain endothelium model was determined using a fluorescent glucose analog. The glucose permeability increased with the increasing incubation time when exposed to blast TBI and PSDs. Accordingly, the disrupted endothelium is creating a hyperglycemic environment for the brain tissue. To determine the effect of hyperglycemia in neurons, cortical neurons were isolated from rats and exposed to hyperglycemic conditions. Detrimental effects of excess glucose to neuron were confirmed by distorted neuron axons visualized by b-III tubulin, aggregation of Caspase-3, and proteolysis of Tau proteins. Interestingly, pre- and post-treatment with (P188+NAC) for 24 hours proved to have therapeutic potential. Examination of (P188+NAC) pre- and post-treated neurons demonstrated a suppression of aggregated Caspase-3 and the cleavage of Tau protein, indicating that the functionality of neurons may be restored. The results of this study indicate that blast TBI and PSDs exacerbates hyperglycemia-induced cortical neuronal injury. The contribution of (P188+NAC) to neuroprotection is significant.

Methods of delivering a therapeutic agent to an injury site in a subject in need thereof are also disclosed. The method can include administering to the subject a therapeutically effective amount of a composition as disclosed herein. in some examples, the composition is administered intranasally, orally, parenterally, subcutaneously, pulmonarily, intravenously, intraperitoneally, intrathecally, intracerbrally, epidurally, intramuscularly, intradermally, or intracarotidly. Preferably, the composition is administered intranasally or orally. In some embodiments, the therapeutic agent when administered alone, exhibits negligible to no oral bioavailability. The composition can be delivered to an injured site in the subject's central nervous system. For example, the composition can be delivered to the blood brain barrier or brain tissue.

Methods of making a composition are also disclosed, the method can include forming a primary emulsion from an aqueous solution of the therapeutic agent and a solution comprising a polyester derived polymer; forming a secondary emulsion by mixing the primary emulsion and a solution comprising a stabilizing polymer, wherein the secondary emulsion comprises drug loaded nanoparticles; separating the drug, loaded nanoparticles; and binding a targeting agent to the drug, loaded nanoparticles via a linker.

Various aspects and features of embodiments of the present disclosure will become further apparent to those skilled in the art upon reviewing the following detailed description and the attached Appendix, which is an integral part of the present Application.

### BRIEF DESCRIPTION OF THE DRAWINGS

FIG. 1 is a graphical table of contents demonstrating the disclosed theragnostic approach. Injuries were simulated by either a mechanical trauma or using an inflammatory factor for comparison purposes. Injured endothelium up-regulates the E-selectin expression that can be targeted by decorating nanoparticles with specific ligands and deliver drugs to the injured endothelial cells to restore the structural and functional integrity of the brain endothelium.

FIGS. 2A-2N shows fluorescent images of time-dependent E- and P-selectin expressions post TNF-α stimulation and in response to microcavitation, and Western blot protein analysis of E-selectin after blasts exposure. Control cells cultured in DMEM with 1% serum showed essentially no P- or E-selectin expression (FIGS. 2A, 2B, 2K). Following a 4-hour stimulation with TNF-α (10 ng/ml), the mouse brain endothelial cells were further incubated in fresh 1% serum-medium for 1 (FIGS. 2C and 2D), 3 (FIGS. 2E and 2F), 6 (FIGS. 2G and 2H), and 24 hours (FIGS. 21 and 2J) hours, and E- and P-selectins were visualized. Following exposure to the blast-induced mechanical trauma, cells were incubated in fresh 1% serum-medium for 1 (FIG. 2L) or 3 hours (FIG. 2M), and E-selectin was immunofluorescently stained. Nuclei were visualized blue using DAPI. Bar=50 mm. (FIG. 2N) Western blot protein analysis showing an up-regulated E-selectin protein level.

FIGS. 3A-3H show the superoxide level was visualized using MitoSox after incubation the cells for 30 min at room temperature and ZO-1 found in the tight junction was imaged. (FIG. 3A) Control cells showing essentially no superoxide expression. (FIG. 3B) A high level of superoxide in response to TNF-α, if left untreated. (FIG. 3C) NAC treatment suppresses the superoxide expression. (FIG. 3D) When treated simultaneously with NAC+P188, the diminished superoxide level remained unchanged. Nuclei were stained with DAPI. Bar=50 mm for the panels A through D. (FIG. 3E) Quantitative analysis of superoxide expressions. Data represent mean±SD. *p<0.05 when compared to the control. n=6. (FIG. 3F) ZO-1 expression in control BECs. (FIG. 3G) Image of disrupted ZO-1 distribution and expression in response to TNF-α. (FIG. 3H) The potential therapeutic efficacy of NAC+P188 is demonstrated in partially restoring tight junction after exposure to an inflammatory disruption. Bar=50 mm for the panels F through H.

FIGS. 4A-4E show TEM images and characterization of PLGA NPs. Transmission electron microscope images of PLGA NPs; (FIG. 4A) blank PLGA NPs, (FIG. 4B) P188-loaded PLGA NPs, (FIG. 4C) NAC-loaded NPs, (FIG. 4D) P188+NAC-loaded PLGA NPs. (FIG. 4E) Characterization of the physical properties of PLGA NPs under different loading conditions. No statistical difference was observed among the NP sizes under 4 loading conditions. Data represent mean±SD, n=6.

FIG. 5A-5B show time-dependent release of NAC or P188. (FIG. 5A) The release of NAC (circles) and that of P188 (squares) from NPs were monitored as a function of time. (FIG. 5B) The first several data points were plotted separately to determine the time required to reach the half of maximum release. Linear regression was used to fit the data to a line originating from the origin. Data represent mean±SD, n=6. Some error bars were smaller than the symbol sizes used to illustrate the release kinetics of two non-biologics.

FIGS. 6A-6H show internalization and targeting efficiency studies of conjugated and unconjugated PLGA NPs. (FIGS. 6A to 6D) Fluorescent images showing the specificity of PSGL-1 conjugated PLGA NPs to either inactivated or TNF-a activated brain endothelial cells. (FIG. 6E) Quantitative analysis of the specificity of PSGL-1 conjugated PLGA NPs. Data represent mean±SD of 3 to 6 independent experiments. *p<0.05 compared to the solid black bar. (FIGS. 6F-6H) Targeting efficiency study of conjugated PLGA NPs to endothelial cells exposed to mechanical trauma. (FIG. 6F) Control cells incubated with PSGL-1 conjugated PLGA NPs for 1 hour. (FIG. 6G) Endothelial cells exposed to mechanical trauma and then incubated with PSGL-1 conjugated PLGA NPs for 1 hour. The area of detached cell is apparent and indicated by a dotted line for better illustration. (FIG. 6H) Quantitative analysis of the specificity of PSGL-1 conjugated PLGA NPs. Data represent mean±SD of 3 to 6 independent experiments. *p<0.05.

FIGS. 7A-7J show closure of mechanically induced lesion. In response to a mechanical trauma, the PLGA NPs loaded with P188+NAC induced proliferation/migration to repair the lesion. Results were compared to positive (P188+NAC free drug) and negative control experiments (no drug). (FIG. 7A to 7C) Injury sites prior to treatment. (FIG. 7D to 7F) After 12 hours of treatment, the lesion appears to have been repopulated with cells. (FIG. 7G to 7I) Magnified images that correspond to the panels D through F. (FIG. 7J) Quantitative analysis of the lesion closure. The conjugated P188+NAC NPs and P188+NAC free drug showed a wound closure that was statistically indistinguishable. Data represent mean±SD. *p<0.05 when compared to the control (n=3).

FIGS. 8A-8B show permeability of brain endothelium. The permeability coefficient (P) of large 10 kDa Dextran was measured. The increase in the permeability is correlated with the mechanical trauma (FIG. 8A) or TNF-a cytokine insult (FIG. 8B). When compared to the control (EC monolayer), the permeability was significantly increased. Treatment of injured cells with the combination of P188+NAC NPs for 12 hours restored the permeability. Data represent mean±SD. *p<0.05, (n=3).

FIGS. 9A-9C show Western blot analysis using Image Lab 6.0.1. following blast (shockwave) exposure.

FIGS. 10A-10E show time-dependent uptake of conjugated PLGA NPs.

FIGS. 11A-11C show cytocompatibility of NPs with brain endothelial cells incubated with blank PLGA NPs (b-PLGA), NAC and NAC+P188 PLGA NPs.

FIGS. 12A-12F are representative phase contrast images of in vitro scratch wound model of brain endothelial cells treated with the combination of P188+NAC.

FIGS. 13A-13D show schematics of blast chamber and a brief flow of experimental design from culture insert, proof of cell adhesion to PETE membrane to blast chamber and finally diffusion chamber. (FIG. 13A) design of the blast chamber, depicting the events that takes place after transferring the cell culture insert to the blast chamber. Microbubbles are formed, and the collapse of the bubbles causes BBE destruction and the formation of a “crater”. (FIG. 13B) cell culture insert. FITC cell tracker showing that PETE membrane supports endothelial cell culture. Endothelial cells were seeded on a fibronectin-coated membrane. (FIG. 13C) diagrammatic representation of the blast chamber. (FIG. 13D) schematic description of the diffusion chamber with a monolayer of cells on the luminal side of the membrane. Permeability was measured by introducing FITC dextran dye of different molecular weights into the luminal chamber and measuring the time-dependent concentration in the abluminal chamber.

FIGS. 14A-14E show characterization of mouse primary brain microvascular endothelial cells (MPBMECs). (FIG. 14A) Phase contrast microscopic view of the MPBMECs showing tightly packed cell organization. (FIG. 14B) Human umbilical vascular endothelial cells (HUVECs) demonstrate a similar morphology. (FIG. 14C) Immunofluorescence staining demonstrating the expression of endothelial cell tight junction-associated marker, ZO-1 after 4 days of culture. (FIG. 14D) Immunofluorescence staining of mouse endothelial cell F-actin. Nuclei counterstained with DAPI. (FIG. 14E) Branched network of capillary-like cords formed by MPBMECs in Matrigel extracellular matrix after 6 hours in EGM-2 medium (X20).

FIGS. 15A-15D show disruption of blood brain endothelium. Immuno-fluorescence staining of endothelial cells for ZO-1, F-actin following microcavitation. (FIGS. 15A & 15B) exposure to one blast and (FIGS. 15C & 15D) exposure to five repetitive blasts. There is no obvious disorganization of tight junctions in response to one blast exposure, Formation of the crater after five blasts exposure is accentuated by dotted circles. The nuclei were stained with DAPI.

FIGS. 16A-16C show schematics of permeability experiment and measurement of permeability coefficient (P). (FIG. 16A) Procedures summarized for the evaluation of Dextran molecule permeability across in vitro BBE model. (FIGS. 16B & 16C) Permeability coefficients measured using 3 and 10 kDa Dextran molecules. The adjusted permeability coefficient was determined according to the procedures in (FIG. 16A). The adjusted permeability coefficients reflect the biotransport properties through tight junctions. *p<0.05 compared the control (EC Monolayer).

FIGS. 17A-17D show dose dependent study of P188 on brain endothelial cell (BECs) viability and modulated permeability coefficient. (FIG. 17A) Cell viability was measured after 24 h with MTT assay (n=3). Asterisks represent a significant difference compared to control (p<0.05). (FIG. 17B) Changes in the permeability coefficients of the monolayer exposed to microcavitation and treated with P188 using 3 and 10 kDa Dextran molecules. *p<0.05 compared to control (EC monolayer). (FIG. 17C) Expansion of the crater if left untreated. Cells were exposed to microcavitation and incubated in serum-free media for 1 h and stained for ZO-1 and nuclei. (FIG. 17D) The crater expansion is prevented if the cells were treated with P188 for 3 hrs following microcavitation. Immunofluorescent images showed reconstitution of the tight junctions in response to P188.

FIGS. 18A-18B show gene expression and Western blot analysis. (FIG. 18A) Change in the ZO-1 gene expression determined from 3 independent experiments. (FIG. 18B) western blot protein analysis showing a recovered ZO-1 protein level in response to the P188 treatment.

FIGS. 19A-19J show activation of MMP-2 & 9 by biochemical disruption of tight junctions. Immunofluorescent staining of MMP-2 & 9 in TNF-a activated cells. ECs were pretreated with P188 for 3 hrs before incubating with TNF-a for 1 hr and then stained for MMP-2 & 9 (FIGS. 19A & 19E), respectively. ECs incubated with TNF-a for 1 hr and then stained for MMP-2 & 9 (FIGS. 19B & 19F), respectively. ECs incubated with TNF-a for 1 hr, treated with P188 for 3 hrs afterward, and then stained for MMP-2 & 9 (FIGS. 19C & 19G), respectively. Results from all groups were compared with control (FIGS. 19D & H). (FIGS. 191 & 19J) The efficacy of P188 in suppressing the gene expression of MMP-2 & 9 in both pre-treated and post-treated groups. Fold change were calculated from triplicate experiments. *p<0.05.

FIGS. 20A-20J show activation of MMP-2 & 9 in response to microcavitation (i.e., mechanical trauma). ECs were pretreated with P188 for 3 hrs before exposure to microcavitation and then stained for MMP-2 & 9 (FIGS. 20A & 20E), respectively. ECs exposed to microcavitation and then stained for MMP-2 & 9 without P188 treatment (FIGS. 20B & 20F), respectively. ECs exposed and treated with P188 for 3 hrs afterward, and then stained for MMP-2 & 9 (FIGS. 20C & 20G), respectively. Results from all groups were compared to control (FIGS. 20D & 20H). Effects of P188 in suppressing the gene expression of MMP-2 & 9 (FIGS. 20I & 20J) in both pre-treated and post-treated groups. Fold change were calculated from triplicate experiments. *p<0.05.

FIGS. 21A-21D show in vitro MMP-2 and 9 enzyme activity and gene expression in response to microcavitation. (FIGS. 21A and 21B) Cells exposed to microcavitation demonstrated a significantly increased MMP-2 and 9 enzymatic activity when compared to the control. This increased activity was inhibited by a doxycycline or phenanthroline pretreatment. *p<0.05. (FIGS. 21C and 21D) Doxycycline downregulated the MMP-2 and -9 gene expressions. A tissue inhibitor of metalloproteinase (phenanthroline) also induced the similar gene downregulation. Cells treated with doxycycline showed the antibiotics acted at the transcriptional level. Results indicate fold change±SD (n=3). *p<0.05.

FIG. 22 shows P188 and doxycycline attenuates endothelial cell permeability. Permeability is significantly increased in cells exposed to microcavitation versus control (EC Monolayer). The increased permeability is significantly inhibited by P188 or doxycycline. The combination of P188 and doxycycline was shown to further restore the permeability observed in control cells. *p<0.05.

FIGS. 23A-23C show internalization of P188. (FIG. 23A) Image of internalized TAMRA fluorophores alone in control cells. Nuclei counterstained with DAPI. The fluorophore diffused into the cytoplasm and nuclei. (FIG. 23B) Immunofluorescent images showing endocytosis of conjugated TAMRA-P188 complexes in control cells. Diffusion of the complex into the nuclei was not evident. (FIG. 23C) Immunofluorescent images showing endocytosis of conjugated TAMRA-P188 complexes in cells exposed to microcavitation. There is evidence that the complexes diffused into the nuclei in some cells (e.g., purple color), suggesting potential reorganization of the nuclear envelope caused by microcavitation.

FIG. 24 shows schematics showing the potential mechanisms underlying the protective effects of P188. Blunt force trauma up-regulates MMP-2 & 9 in the brain endothelium which, in turn, degrades the tight junction structure and integrity and lead to an increased BBB permeability. The BBB protection by P188 against traumatic brain injury is proposed to be mediated by down-regulation of MMP-2 & 9, which inhibits the degradation of tight junction, and thereby restores the biotransport properties of the brain endothelium. Collectively, P188 attenuates the extent of injuries caused by brain traumas.

### DESCRIPTION

The disclosed subject matter can be understood more readily by reference to the following detailed description, the Figures, and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

It is understood that the disclosed methods and systems are not limited to the particular methodology, protocols, and systems described as these may vary. It is also to be understood that the terminology used herein is for the purpose of describing particular embodiments only, and is not intended to limit the scope of the present invention which will be limited only by the appended claims.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “optional” or “optionally” means that the subsequently described event or circumstance can or cannot occur, and that the description includes instances where said event or circumstance occurs and instances where it does not.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a patient. A patient refers to a subject afflicted with an injury, disease or disorder, or inflammation such as, for example, a traumatic brain injury. The term “patient” includes human and veterinary subjects. In an aspect, the subject has been diagnosed with a need for treatment for traumatic brain injury.

The terms “treating”, “treatment”, “therapy”, and “therapeutic treatment” as used herein refer to the medical management of a subject or a patient with the intent to cure, ameliorate, or stabilize a disease, pathological condition, or disorder, such as, for example, traumatic brain injury. Beneficial or desired clinical results include, but are not limited to, any one or more of: alleviation of one or more symptoms, diminishment of extent of the injury, stabilized (i.e., not worsening) state of injury, preventing or delaying further injury, delaying occurrence or recurrence of injury, delay or slowing of injury progression, amelioration of the injury state (including general symptoms), and remission (whether partial or total). In addition, this term includes palliative treatment, that is, treatment designed for the relief of symptoms rather than the curing of the disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) inhibiting the disease, i.e., arresting its development; or (ii) relieving the disease, i.e., causing regression of the disease. In an aspect, the condition is an injury or inflammation for example, due to trauma. In an aspect, the injury can be any traumatic injury known to the art.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed. For example, in an aspect, preventing can refer to the preventing of chronic or secondary injury of endothelial cells.

As used herein, the term “diagnosed” means having been subjected to a physical examination by a person of skill, for example, a physician or a researcher, and found to have a condition that can be diagnosed or treated by compositions or methods disclosed herein. For example, “diagnosed with traumatic brain injury” means having been subjected to a physical examination by a person of skill, for example, a physician or a researcher, and found to have a condition that can be diagnosed or treated by a compound or composition that alleviates or ameliorates the injury.

As used herein, the terms “administering” and “administration” refer to any method of providing a composition to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a condition.

The term “contacting” as used herein refers to bringing a disclosed composition or pharmaceutical preparation and a cell, target receptor, or other biological entity together in such a manner that the compound can affect the activity of the target (e.g., receptor, transcription factor, cell, etc.), either directly; i.e., by interacting with the target itself, or indirectly; i.e., by interacting with another molecule, co-factor, factor, or protein on which the activity of the target is dependent.

As used herein, the term “determining” can refer to measuring or ascertaining a quantity or an amount or a change in expression and/or activity level.

As used herein, the terms “effective amount” and “amount effective” refer to an amount that is sufficient to achieve the desired result or to have an effect on an undesired condition. For example, in an aspect, an effective amount of the polymeric nanoparticle is an amount that mitigates disruption of brain endothelial cells such as by alleviating the loss of tight junctions. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

By “modulate” is meant to alter, by increase or decrease. As used herein, a “modulator” can mean a composition that can either increase or decrease the expression level or activity level of a gene or gene product such as a peptide. Modulation in expression or activity does not have to be complete. For example, expression or activity can be modulated by about 10%, 20%, 30%, 40%, 50%, 60%, 70%, 80%, 90%, 95%, 99%, 100% or any percentage in between as compared to a control cell wherein the expression or activity of a gene or gene product has not been modulated by a composition.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The term “drug-loaded nanoparticles” as used herein refers to nanoparticles comprising a pharmacologically active substance. A pharmacologically active substance can either be a therapeutic agent or a diagnostic agent. Hence the “drug-loaded nanoparticles” of the disclosure comprise at least one therapeutic agent and/or at least one diagnostic agent being absorbed to, adsorbed to, or incorporated into said nanoparticles.

The term “blood-brain barrier” as used herein refers to the blood-brain barrier as such, i.e. the endothelium of the brain vessels, the basal membrane and neuroglial cells. The blood-brain barrier serves to control the transfer of substances into the brain. The term “blood-brain barrier” as used herein refers to the blood-spinal barrier and also to the blood-retina barrier.

The term “brain injury” refers to a condition in which the brain is damaged by injury caused by an event. As used herein, an “injury” is an alteration in cellular or molecular integrity, activity, level, robustness, state, or other alteration that is traceable to an event. For example, an injury includes a physical, mechanical, chemical, biological, functional, infectious, or other modulator of cellular or molecular characteristics. An event can include a physical trauma such as an impact (percussive or concussive) or a biological abnormality such as a stroke resulting from either blockade or leakage of a blood vessel. An event is optionally an infection by an infectious agent. A person of skill in the art recognizes numerous equivalent events that are encompassed by the terms injury or event.

According to some embodiments of the method of the present disclosure, a healthy subject's predisposition to future onset of brain injury is also diagnosed. According to some embodiments said predisposition is due to previous injury or due to family history.

More specifically, the term “brain injury” refers to a condition that results in central nervous system damage, irrespective of its pathophysiological basis. Among the most frequent origins of a “brain injury” are stroke and traumatic brain injury (TBI). A “stroke” is classified into hemorrhagic and non-hemorrhagic. Examples of hemorrhagic stroke include cerebral hemorrhage, subarachnoid hemorrhage, and intracranial hemorrhage secondary to cerebral arterial malformation, while examples of non-hemorrhagic stroke include cerebral infarction.

The term “brain injury” also refers to subclinical brain injury, spinal cord injury, and anoxic-ischemic brain injury. The term “subclinical brain injury” (SCI) refers to brain injury without overt clinical evidence of brain injury. A lack of clinical evidence of brain injury when brain injury actually exists could result from degree of injury, type of injury, level of consciousness, medications particularly sedation and anesthesia. Many of these origins can lead to Chronic Traumatic Encephalopathy (CTE).

The term “traumatic brain injury” refers to a brain injury resulting from direct or indirect shock load or loads applied to the brain causing it to move rapidly and unnaturally within a patient's skull and include, but not be limited to, brain injuries caused by: (a) objects penetrating the skull, such as, bullets, arrows, and other physical objects which pass through the skull and enter the brain, (b) impact loads applied to the head or other portions of the patient's body, (c) surgically induced trauma, (d) explosions, such as might exist in warfare, through impacting of grenades, bombs, and other explosives, which cause substantial tremors in the earth in relatively-close proximity to where an individual is standing, as well as similar tremors created by nonexplosive means, such as sports injuries, vehicular accidents, collapse of buildings and earthquakes, for example. The results of traumatic brain injury may be of various types, but in each instance, will involve temporary or permanent reduction in the ability of the brain to function normally and may cause death.

One of the consequences of a traumatic brain injury frequently is the generation of inflammation within the brain as the shock to the brain serves to increase the permeability of the endothelial cells, thereby permitting loss of fluids from the vascular structure into the brain. Such a leakage frequently occurs due to the increased porosity of the blood vessels resulting from the trauma, thereby causing blood serum to leak through the vessels into the brain area. As this builds up, this can generate inflammation and swelling of the brain, which may require surgical intervention.

Clinically, traumatic brain injury can be rated as mild, moderate or severe based on TBI variables that include duration of loss of consciousness (LOC), Glasgow Coma Score (GCS) and post-traumatic stress amnesia.

As used herein, “secondary brain trauma” refers to damage to the brain of a patient post-acute brain injury, i.e., during the secondary injury phase of a TBI.

The term “brain injury biomarker” refer to a biomolecule such as a protein, including those described herein, that can be used to diagnose brain injury in a patient. Brain injury biomarker proteins include, but are not limited to, cell adhesion molecules including P-selectin, E-selectin, and L-selectin; SNCB, GFAP, S100B, MT3, ICAM5, BDNF, and/or NSE. The term also includes other brain injury biomarker proteins known in the art including neurogranin (NRGN), myelin basic protein (MBP), PAD-2, tubulin beta-4B chain, tubulin alpha-IB chain, CNPase, PPIA, Septin-7, Elongation factor 1-alpha2, TPPP, TPPP3, Ermin Isoform 2, NDRG2 Isoform 2, astrotactin 1 (ASTN1), brain angiogenesis inhibitor 3 (BAD); carnosine dipeptidase 1 (CNDP 1); ERMTN; glutamate receptor metabotropic 3 (GRM3); kelch-like protein 32 (KLH32); melanoma antigen family E,2 (MAGE2); neuregulin 3 (NRG3); oligodendrocyte myelin glycoprotein (OMG); solute carrier family 39 (zinc transporter); reticulon 1 (RTN1); and peptidylarginine deiminase (types 1-4 and 6) (PAD). In addition, the term “brain injury biomarkers” also includes the isoforms and/or post-translationally modified forms of any of the foregoing. The disclosure contemplates the detection, measurement, quantification, determination and the like of both unmodified and modified (e.g., citrullination or other post-translational modification) proteins/polypeptides/peptides as well as autoantibodies to any of the foregoing. In certain embodiments, it is understood that reference to the detection, measurement, determination, and the like, of a biomarker refers detection of the protein/polypeptide/peptide (modified and/or unmodified). In other embodiments, reference to the detection, measurement, determination, and the like, of a biomarker refers detection of autoantibodies of the protein/polypeptide/peptide.

A weight percent (wt. %) of a component, unless specifically stated to the contrary, is based on the total weight of the formulation or composition in which the component is included.

It is understood that throughout this specification the identifiers “first” and “second” are used solely to aid in distinguishing the various components and steps of the disclosed subject matter. The identifiers “first” and “second” and the like are not intended to imply any particular order, amount, preference, or importance to the components or steps modified by these terms.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

Reference will now be made in detail to specific aspects of the disclosed materials, compounds, compositions, articles, and methods, examples of which are illustrated in the accompanying Examples.

Nanoparticle Compositions

Disclosed herein are compositions and methods for diagnosis and therapy through targeted nano-delivery to injured brain endothelium. The compositions can be derived from engineered polymeric nanoparticles that encapsulate therapeutic non-biologics and decorated with ligands to specifically target injured endothelium such as the E-selectin. The properties of the polymeric nanoparticles can be tunable, thereby facilitating a targeted drug delivery strategy. The strategy described herein can be used to provide a theragnostic approach for treatment of modulated brain endothelium associated with TBI. These and other materials are disclosed herein, and it is understood that when combinations, subsets, interactions, groups, etc. of these materials are disclosed that while specific reference of each various individual and collective combinations and permutation of these compositions cannot be explicitly disclosed, each is specifically contemplated and described herein. This concept applies to all aspects of this application including, but not limited to, steps in methods of making and using the compositions disclosed herein. Thus, if there are a variety of additional steps that can be performed it is understood that each of these additional steps can be performed with any specific embodiment or combination of embodiments of the methods disclosed herein.

Drug Loaded Nanoparticle Compositions

In some aspects of the disclosed compositions, the compositions can be formulated as polyester derived nanoparticles, wherein the nanoparticles comprise a therapeutic agent encapsulated therein and a targeting ligand bound to a surface of the nanoparticle. The disclosed nanoparticles can preserve particle integrity, demonstrating the potent utility of this approach for targeted drug delivery. Importantly, the nanoparticles can act as therapeutic drug transporters and neither degrade nor modify drug-loaded particles in transit. As such, the therapeutic drug(s) are delivered unaltered to its intended target site(s).

In one aspect, disclosed herein are a population of polyester derived nanoparticles; wherein each polyester derived nanoparticle comprises a) a therapeutic agent encapsulated therein for treating traumatically injured, inflamed, diseased, or disrupted endothelial cells, and b) a targeting ligand bound to the nanoparticle, wherein the targeting ligand binds to a biomarker for the injured, inflamed, diseased, or disrupted endothelial cells.

The polyester derived nanoparticle preferably comprises a biocompatible polyester or copolyester. In some embodiments, the nanoparticles are derived from polylactides (PLA), also called polylactic acids. Polylactides are polyesters on the basis of lactic acid. Polylactic acids are polyhydroxyacids. They are biocompatible and biodegradable. The properties of polylactides depend primarily on their molecular weight, degree of crystallinity, and the portion of copolymers, if applicable. The glass transition temperature, the melting temperature, the tensile strength and the E-module of the polylactides increase as the molecular weight of the polylactides increases.

In some examples, the weight average molecular weight of the polyester derived nanoparticle can be 5,000 Da or greater, 7,500 Da or greater, 10,000 Da or greater, 12,500 Da or greater, 15,000 Da or greater, 17,500 Da or greater, 20,000 Da or greater, 22,000 Da or greater, 24,000 Da or greater, 25,000 Da or greater, 30,000 Da or greater, 32,500 Da or greater, 35,000 Da or greater, 40,000 Da or greater, 45,000 Da or greater, 50,000 Da or greater, 60,000 Da or greater, or 70,000 Da or greater. In some examples, the weight average molecular weight of the polyester derived nanoparticle can be 250,000 Da or less, 200,000 Da or less, 150,000 Da or less, 120,000 Da or less, 100,000 Da or less, 90,000 Da or less, 80,000 Da or less, 70,000 Da or less, 60,000 Da or less, 50,000 Da or less, 45,000 Da or less, 40,000 Da or less, 37,500 Da or less, 35,000 Da or less, 32,500 Da or less, 30,000 Da or less, 27,500 Da or less, or 25,000 Da or less. In some examples, the weight average molecular weight of the polyester derived nanoparticle can be 5,000 to 100,000, from 10,000 to 50,000, from 20,000 to 35,000, or from 24,000 to 30,000.

Polylactides can be obtained by ring-opening polymerization of lactide. The ring-opening polymerization is performed at temperatures between 140 and 180° C. in the presence of stannous octoate catalyst. Polylactides with high molecular weight can be easily produced by this method. In addition, high molecular weight and pure polylactides can be generated directly from lactic acid by the so-called polycondensation.

In some embodiments, the nanoparticles are derived from polylactide coglycolides (PLGA), which are biodegradable polymers that comprise lactic acid linked with glycolic acid. The respective percentages of lactic acid and glycolic acid in the PLGA nanoparticles may play a role in the rate of drug release. In some instances, the ratio of lactide to glycolide can be from 90:10 to 10:90, such as from 20:80 to 80:20, from 30:70 to 70:30, from 40:60 to 60:40, from 45:55 to 55:45, or 50:50. Lactide is optically active, and any proportions of D and L isomers may be present, ranging from pure D-lactide to pure L-lactide, with racemates comprising 50% D-lactide and 50% L-lactide.

Other suitable polymers that can be included in the nanoparticle include, without limitation, poly (2-oxazoline) amphiphilic block copolymers, polyethylene glycol-polylactic acid (PEG-PLA), PEG-PLA-PEG, polyethylene glycol-poly(lactide-co-glycolide) (PEG-PLG), polyethylene glycol-poly(lactic-co-glycolic acid) (PEG-PLGA), polyethylene glycol-polycaprolactone (PEG-PCL), polyethylene glycol-polyaspartate (PEG-PAsp), polyethylene glycol-poly(glutamic acid) (PEG-PGlu), polyethylene glycol-poly(acrylic acid) (PEG-PAA), polyethylene glycol-poly(methacrylic acid) (PEG-PMA), polyethylene glycol-poly(ethyleneimine) (PEG-PEI), polyethylene glycol-poly(L-lysine) (PEG-PLys), polyethylene glycol-Poly(2-(N,N-dimethylamino) ethyl methacrylate) (PEG-PDMAEMA), and polyethylene glycol-chitosan derivatives.

The nanoparticles suitable for use herein comprise at least one targeting agent and at least one therapeutic agent, i.e., the therapeutic agent for treating traumatically injured, inflamed, diseased, or disrupted endothelial cells.

The drug loaded nanoparticle can be up to 1 m in diameter, for example, from about 10 nm to about 500 nm in diameter, from about 50 nm to about 500 nm in diameter, from about 100 nm to about 400 nm in diameter, from about 200 nm to about 500 nm in diameter, or from about 200 nm to about 350 nm in diameter. In some embodiments, the drug loaded nanoparticle as used in the present disclosure have an average particle size ranging from about 10 nm to about 1000 nm, for example from about 100 nm to about 1000 nm, from about 200 nm to about 1000 nm, from about 300 nm to about 1000 nm, from about 400 nm to about 1000 nm, from about 500 nm to about 1000 nm, from about 600 nm to about 1000 nm, from about 700 nm to about 1000 nm, from about 800 nm to about 1000 nm, from about 900 nm to about 1000 nm, from about 10 nm to about 900 nm, from about 100 nm to about 900 nm, from about 200 nm to about 900 nm, from about 300 nm to about 900 nm, from about 400 nm to about 900 nm, from about 500 nm to about 900 nm, from about 600 nm to about 900 nm, from about 700 nm to about 900 nm, from about 800 nm to about 900 nm, from about 10 nm to about 800 nm, from about 100 nm to about 800 nm, from about 200 nm to about 800 nm, from about 300 nm to about 800 nm, from about 400 nm to about 800 nm, from about 500 nm to about 800 nm, from about 600 nm to about 800 nm, from about 700 nm to about 800 nm, from about 10 nm to about 700 nm, from about 100 nm to about 700 nm, from about 200 nm to about 700 nm, from about 300 nm to about 700 nm, from about 400 nm to about 700 nm, from about 500 nm to about 700 nm, from about 600 nm to about 700 nm, from about 10 nm to about 600 nm, from about 100 nm to about 600 nm, from about 200 nm to about 600 nm, from about 300 nm to about 600 nm, from about 400 nm to about 600 nm, from about 500 nm to about 600 nm, from about 10 nm to about 500 nm, from about 100 nm to about 500 nm, from about 200 nm to about 500 nm, from about 300 nm to about 500 nm, from about 400 nm to about 500 nm, from about 10 nm to about 400 nm, from about 100 nm to about 400 nm, from about 200 nm to about 400 nm, from about 300 nm to about 400 nm, from about 10 nm to about 300 nm, from about 100 nm to about 300 nm, from about 200 nm to about 300 nm, from about 10 nm to about 200 nm, from about 100 nm to about 200 nm, or from about 10 nm to about 100 nm.

The polydispersity of the population of drug loaded nanoparticles can be 0.3 or less, 0.25 of less, 0.2 or less, 0.15 or less, 0.1 or less, 0.08 or less, or 0.05 or less.

In some embodiments, the nanoparticles have a loading efficiency of the therapeutic agent of greater than 50%. In some embodiments, the therapeutic agent (for example, the surfactant) and the nanoparticles can be present in a weight ratio 5:1 or greater, 10:1 or greater, 20:1 or greater, 30:1 or greater, 40:1 or greater, 50:1 or greater, 60:1 or greater, 70:1 or greater, 80:1 or greater, 100:1 or greater, from 50:1 to 500:1, from 100:1 to 500:1, or from 150:1 to 400:1.

The nanoparticles can be rod shaped, irregular, or round shaped. The nanoparticles can be neutral or charged. The nanoparticles can be charged positively or negatively.

Therapeutic Agent

As described herein, the nanoparticles can comprise at least one therapeutic agent, i.e., a therapeutic agent for treating traumatically injured, inflamed, diseased, or disrupted endothelial cells. In one aspect, the nanoparticles comprise one or more therapeutic agents for treating traumatic brain injury. For example, the nanoparticles can comprise one or more therapeutic agents for mitigating brain endothelial cell disruption, such as by alleviating the loss of tight junctions.

In some examples, the therapeutic agent comprises at least one surfactant. Surfactants have been shown to reseal damaged cell membrane and facilitate repair of injured endothelial cells. The surfactant is preferably biocompatible organic compounds that are amphiphilic. In a particular example, the surfactant is an amphiphilic block copolymer. In another example, at least one surfactant of the nanoparticle is an amphiphilic block copolymer, particularly a copolymer comprising at least one block of poly (oxyethylene) and at least one block of poly (oxypropylene). The surfactant can be charged or neutral. In a particular example, the surfactant is positively or negatively charged, particularly negatively charged.

In some examples, the therapeutic agent can include a poloxamer surfactant. Poloxamers are nonionic polyoxyethylene-polyoxypropylene block co-polymers with the general formula HO(C₂H₄O)ₐ(—C₃H₆O)_{b}(C₂H₄O)ₐH. They are available in different grades which vary from liquids to solids. Poloxamers are used as emulsifying agents, solubilizing agents, surfactants, and as wetting agents for antibiotics.

In a particular example, the surfactant can include at least one block of poly (oxyethylene) and at least one block of poly (oxypropylene). Amphiphilic block copolymers are exemplified by the block copolymers having the formulas:

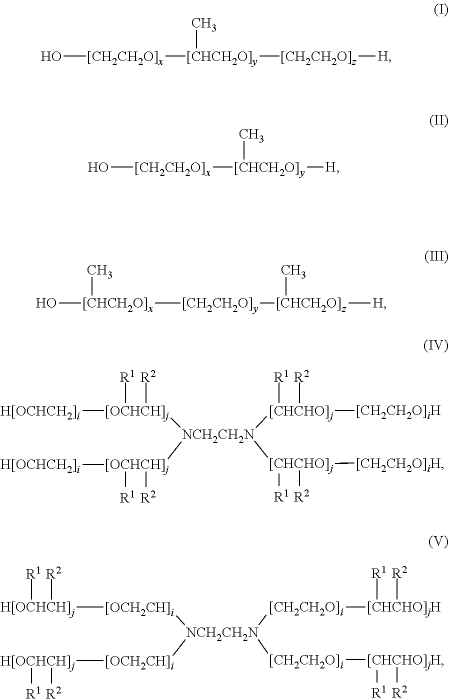

in which x, y, z, i, and j have values from about 2 to about 800, particularly from about 5 to about 200, more particularly from about 5 to about 80, and wherein for each R¹, R² pair, as shown in formula (IV) and (V), one is hydrogen and the other is a methyl group. The ordinarily skilled artisan will recognize that the values of x, y, and z will usually represent a statistical average and that the values of x and z are often, though not necessarily, the same. Formulas (I) through (III) are oversimplified in that, in practice, the orientation of the isopropylene radicals within the B block will be random. This random orientation is indicated in formulas (IV) and (V), which are more complete. A number of such compounds are commercially available under such generic trade names as “lipoloxamers,” “PLURONICS™,” “poloxamers,” and “synperonics.” PLURONIC™ copolymers within the B-A-B formula, as opposed to the A-B-A formula typical of PLURONICS™, are often referred to as “reversed” PLURONICS™ “PLURONIC™ R” or “meroxapol.” Generally, block copolymers can be described in terms of having hydrophilic “A” and hydrophobic “B” block segments. Thus, for example, a copolymer of the formula A-B-A is a triblock copolymer consisting of a hydrophilic block connected to a hydrophobic block connected to another hydrophilic block. The “polyoxamine” polymer of formula (IV) is available from BASF under the tradename TETRONIC™. The order of the polyoxyethylene and polyoxypropylene blocks represented in formula (IV) can be reversed, creating TETRONIC R™, also available from BASF.

Polyoxypropylene-polyoxyethylene block copolymers can also be used with hydrophilic blocks comprising a random mix of ethylene oxide and propylene oxide repeating units. To maintain the hydrophilic character of the block, ethylene oxide can predominate.

Similarly, the hydrophobic block can be a mixture of ethylene oxide and propylene oxide repeating units. Such block copolymers are available from BASF under the tradename PLURADOT™. Poly(oxyethylene)-poly(oxypropylene) block units making up the first segment need not consist solely of ethylene oxide. Nor is it necessary that all of the B-type segment contain solely of propylene oxide units. Instead, in the simplest cases, for example, at least one of the monomers in segment A can be substituted with a side chain group.

A number of poloxamer copolymers are designed to meet the following formula:

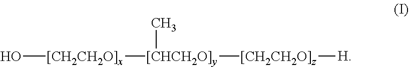

Examples of poloxamers include, without limitation, PLURONIC™ L31, L35, F38, L42, L43, L44, L61, L62, L63, L64, P65, F68, L72, P75, F77, L81, P84, P85, F87, F88, L92, F98, 101, P103, P104, P105, F108, L121, L122, L123, F127, 10R5, 10R8, 12R3, 17R1, 17R2, 17R4, 17R8, 22R4, 25R1, 25R2, 25R4, 25R5, 25R8, 31R1, 31R2, and 31R4. PLURONIC™ block copolymers are designated by a letter prefix followed by a two or a three digit number. The letter prefixes (L, P, or F) refer to the physical form of each polymer, (liquid, paste, or flakeable solid). The numeric code defines the structural parameters of the block copolymer. The last digit of this code approximates the weight content of EO block in tens of weight percent (for example, 80% weight if the digit is 8, or 10% weight if the digit is 1). The remaining first one or two digits encode the molecular mass of the central PO block. To decipher the code, one should multiply the corresponding number by 300 to obtain the approximate molecular mass in daltons (Da). Therefore Pluronic nomenclature provides a convenient approach to estimate the characteristics of the block copolymer in the absence of reference literature. For example, the code “F127” defines the block copolymer, which is a solid, has a PO block of 3600 Da (12×300) and 70% weight of EO. The precise molecular characteristics of each PLURONIC™ block copolymer can be obtained from the manufacturer.

In a particular embodiment, the therapeutic agent includes a surfactant selected from poloxamer 188, poloxamer 185, poloxamer 235, poloxamer 407, polyvinyl alcohol (PVA), 1,2-distearoyl-phosphatidyl ethanolamine-methyl-polyethyleneglycol conjugate-2000 (mPEG₂₀₀₀DSPE), sodium dodecyl sulfate (SDS), and 1,2-dioleoyloxy-3-trimethylammoniumpropane (DOTAP). Poloxamer 188 (PLURONIC® F68 (BASF Corp.)) is a difunctional block copolymer surfactant terminating in primary hydroxyl groups. It is a non-ionic surfactant being relatively non-toxic. Poloxamer 188 has an average molecular weight of 8,400, a viscosity of 1,000 cps at 77° C., a cloud point (10% aqueous) of >100° C., and a HLB value of >24. Poloxamer 185 (PLURONIC® P65 (BASF Corp.)) is a difunctional block copolymer surfactant terminating in primary hydroxyl groups. It is a non-ionic surfactant being relatively non-toxic. PLURONIC® P65 has an average molecular weight of 3,400, a viscosity of 180 cps at 60° C., a cloud point (10% aqueous) of 80-84° C., and a HLB value of 12-18. PLURONIC® P85 (BASF Corp.), also designated as poloxamer 235, is a difunctional block copolymer surfactant terminating in primary hydroxyl groups. It is a non-ionic surfactant being relatively non-toxic. PLURONIC® P85 has an average molecular weight of 4,600, a viscosity of 310 cps at 60° C., a cloud point (10% aqueous) of 83-89° C., and a HLB value of 12-18.

Other biocompatible amphiphilic surfactants that can be used as therapeutic agents or co-agents include, without limitation, poly (2-oxazoline) amphiphilic block copolymers, polyethylene glycol-polylactic acid (PEG-PLA), PEG-PLA-PEG, polyethylene glycol-poly (lactide-co-glycolide) (PEG-PLG), polyethylene glycol-poly (lactic-co-glycolic acid) (PEG-PLGA), polyethylene glycol-polycaprolactone (PEG-PCL), polyethylene glycol-polyaspartate (PEG-PAsp), polyethylene glycol-poly (glutamic acid) (PEG-PGlu), polyethylene glycol-poly(acrylic acid) (PEG-PAA), polyethylene glycol-poly (methacrylic acid) (PEG-PMA), polyethylene glycol-poly (ethyleneimine) (PEG-PEI), polyethylene glycol-Poly(L-lysine) (PEG-PLys), polyethylene glycol-poly (2-(N,N-dimethylamino) ethyl methacrylate) (PEG-PDMAEMA) and polyethylene glycol-chitosan derivatives. Further surfactants can include polysorbate 80 (polyoxyethylene-sorbitan-monooleate, TWEEN® 80), which is a non-ionic surfactant. Polysorbate 80 has an average molecular weight of 1,300, a viscosity of 375-480 mPa s at 25° C., and a HLB value of 14-16. TPGS (D-α-tocopheryl polyethylene glycol 1000 succinate), which is a water-soluble derivative of d-α-tocopheryl succinate may also be used. TPGS is used as a water-soluble delivery form of vitamin E for persons with fat malabsorption syndromes, such as chronic childhood cholestasis. It is also used as an absorption and bioavailability enhancer for the water-insoluble HIV protease inhibitor amprenavir and fat-soluble vitamins such as vitamin D.

In some embodiments, the nanoparticles have a loading efficiency of the surfactant of greater than 50%. In some embodiments, the surfactant and the nanoparticles can be present in a weight ratio 5:1 or greater, 10:1 or greater, 20:1 or greater, 30:1 or greater, 40:1 or greater, 50:1 or greater, 60:1 or greater, 70:1 or greater, 80:1 or greater, 100:1 or greater, from 50:1 to 500:1, from 100:1 to 500:1, or from 150:1 to 400:1.

The nanoparticle can include one or more additional therapeutic agents for treating traumatically injured, inflamed, diseased, or disrupted endothelium. In some examples, the nanoparticles can include an antioxidant. Suitable antioxidants include N-acetylcysteine, glutathione N-acetylcysteine amide, or a physiologically or pharmaceutically acceptable derivative or salt or ester thereof. N-acetylcysteine and glutathione N-acetylcysteine amide provide protective effects against cell damage in its role as a scavenger of free radicals. The nanoparticle composition embraces compositions comprising an antioxidant for protecting cells and tissues from trauma-induced oxidative stress. The systemic damage observed following a traumatic injury is partially due to the overproduction of reactive oxygen species (ROS), which disrupt the delicate pro-oxidant/antioxidant balance of tissues leading to protein, lipid and DNA oxidation.

Additional therapeutic agents that can be included in the nanoparticle composition include doxycycline, chromium III chloride, magnesium sulfate, thiamine, mineral oil, glyceryl stearate, propylene glycol, a hydroxypyridin-2-one or a hydroxamate residue, vitamin C, melatonin, alpha lipoic acid, chlorogenic acid, green tea extract, plant polyphenols, aspirin, quercetin, EGCG, inositol, curcumin, berin, or a combination thereof.

In some embodiments, the nanoparticles have a loading efficiency of the additional therapeutic agent (such as a small molecule antioxidant) of greater than 5%. In some embodiments, the antioxidant (such as NAC) and the nanoparticles can be present in a weight ratio 5:1 or greater, 10:1 or greater, 20:1 or greater, 30:1 or greater, 40:1 or greater, 50:1 or greater, from 5:1 to 100:1, from 10:1 to 100:1, or from 25:1 to 60:1.

Targeting Ligand

As described herein, the nanoparticle can be linked to a targeting ligand. A targeting ligand is a compound that will specifically bind to a specific type of tissue or cell type. In some aspects of the compositions disclosed herein, the drug-loaded polyester derived nanoparticles are for use to target the drug across the blood-brain barrier to the central nervous system, and for treating conditions of the central nervous system or for the manufacturing of a medicament for treating conditions of the central nervous system. The targeting ligand can be selected from an antibody, a small molecule, a peptide, a carbohydrate, an siRNA, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof.

In some aspects, the targeting ligand is a ligand for an endothelial cell surface marker/receptor. The targeting ligand can be a protein, an antibody or fragment thereof immunologically specific for a cell surface marker (e.g., protein or carbohydrate) preferentially or exclusively expressed on the targeted tissue or cell type. In some embodiments, the targeting ligand can be a non-biological material that binds to a cell surface marker (e.g., protein or carbohydrate) preferentially or exclusively expressed on the targeted tissue or cell type. The targeting ligand can be linked directly to the nanoparticle or via a linker, or can be adsorbed to the nanoparticle. The linker is generally a chemical moiety comprising a covalent bond or a chain of atoms that covalently attaches the ligand to the nanoparticle. The linker can be linked to any synthetically feasible position of the ligand and the nanoparticle. Exemplary linkers can comprise at least one optionally substituted; saturated or unsaturated; linear, branched or cyclic alkyl group or an optionally substituted aryl group. The linker can also be a polypeptide (e.g., from about 1 to about 10 amino acids, particularly about 1 to about 5). The linker can be non-degradable and can be a covalent bond or any other chemical structure, which cannot be substantially cleaved or cleaved at all under physiological environments or conditions.

In a particular embodiment, the targeting ligand can be a molecule that specifically target the injured, inflamed, diseased, or disrupted tissue (particularly endothelial cells), wherein the nanoparticle can induce repair and restore the tissue's structural and functional integrity. In some examples, the targeting ligand is a molecule that can bind to a biomarker for injured endothelial cells. P-selectin glycoprotein ligand-1 (PSGL-1), for example, is a glycoprotein found on white blood cells and endothelial cells. PSGL-1 can bind to all three members of the family (P-selectin, E-selectin, and L-selectin) that is part of the broader family of cell adhesion molecules. P-selectin expression peaks relatively early after activation (˜10 min), while the E-selectin expression continues to increase even after 6 hours after activation. Therefore, conjugating NPs with PSGL-1 is expected to lead to an early phase binding of the NPs to the cells via P-selectin, followed by more sustained binding to E-selectin. As demonstrated herein, the targeting of the nanoparticles to P-selectin or E-selectin provides for central nervous system targeting (e.g., brain targeting) decreased toxicity, and prolonged half-life compared to free drug or non-targeted nanoparticles.

In other examples, the targeting ligand can include a non-biologic material, such as a small molecule.

The disclosed nanoparticle formulations can be administered, without limitation parenterally, subcutaneously, orally, topically, pulmonarily, rectally, vaginally, intravenously, intraperitoneally, intrathecally, intracerbrally, epidurally, intramuscularly, intranasally, intradermally, or intracarotidly. In a particular example, the nanoparticles are administered intravenously or intraperitoneally.

Additional Compositions and Methods

While the benefit of combining a therapeutic surfactants and an antioxidant has been demonstrated herein, other drug therapies besides surfactants and antioxidants can be included in the nanoparticles. Disclosed herein are nanoparticle compositions that comprise therapeutic agents for treating a traumatically injured, inflamed, or disrupted endothelial cells.

In an aspect, the disclosed nanoparticles can comprise a functionalizing group that can be used to attach targeting ligands, therapeutics, or imaging agents. The functionalizing groups can be substituents on R¹ and/or R². Examples of suitable functionalizing groups that can be present on the disclosed nanoparticles are azides, amines, alcohols, esters, aldehydes, and the like. In a specific aspect, disclosed are HBPE nanoparticles with these functionalizing groups, in particular azides. In an aspect, the nanoparticles can comprise a targeting ligand. In an aspect, the nanoparticles are conjugated with one or more targeting ligands. In an aspect, the targeting ligand is at high density. In an aspect, the targeting ligand is at low density. In an aspect, the targeting ligand is at high valency. In an aspect, the targeting ligand is at low valency. In an aspect, the targeting ligand is a substrate for a brain endothelial cell.

In some aspect, the nanoparticles can comprise an imaging compound. For example, the imaging compound can be a X-ray, MRI, or PET detectable compound. For example, the imaging compound can comprise a superparamagnetic compound comprising a metal, such as Au, Ag, Pd, Pt, Cu, Ni, Co, Fe, Mn, Ru, Rh, Os, and Ir. In other examples, the imaging compound can be a superparamagnetic compound comprising a metal oxide, such as zinc oxide, titanium dioxide, iron oxide, silver oxide, copper oxide, aluminum oxide, bismuth oxide, and silicon dioxide. In other examples, the imaging compound can be a paramagnetic compound comprising transition metals and lanthanides of groups 1b, 2b, 3a, 3b, 4a, 4b, 5b, 6b, 7b, and 8. In certain examples, the imaging compound can comprise a paramagnetic compound comprising gadolinium (Gd), dysprosium (Dy), chromium (Cr), or manganese (Mn). In other examples the imaging compound can be a radionuclide for PET imaging. For example, the imaging compound can comprise ⁹⁰Y, ¹⁷⁷Lu, ¹⁸F, ⁶⁴Cu, ⁶⁷Cu, ⁸⁹Zr, ¹¹¹In, ¹²⁴I, ¹²³I, and ⁹⁹mTc. In specific examples, the radionuclide that is chelated to the disclosed compounds is ²²⁵Ac, ⁵⁷La, ^{67/69}Ga, ⁶⁸Ga, or ¹⁵²Eu. The radionuclides can be conjugated to compounds such as 1,4,7,10-tetraazacyclo-dodecane-1,4,7,10-tetraacetic acid (DOTA), such as DTPA (diethylene triamine pentaacetic acid), DOTP (1,4,7,10-Tetraazacyclododecane-1,4,7,10-tetra(methylene phosphonic) acid), DOTMA, (1R, 4R, 7R, 10R)-α′α″α′″-Tetramethyl-1,4,7,10-tetraazacyclododecane-1,4,7,10-tetraacetic acid) tetrasodium salt, TETA, (1,4,8,11-Tetraazacyclotetradecane-1,4,8, 11-tetraacetic acid), DOTAM (1,4,7,10-Tetrakis(carbamoylmethyl)-1,4,7,10-tetraazacyclododecane), CB-TE2A (1,4,8,11-tetraazabicyclo[6.6.2]hexadecane-4, 11-diacetic acid), and NOTA ((1,4,7-triazacyclononane-N,N′,N″-triacetic acid). In further examples the imaging compound can be an imaging compound for X-ray/CT imaging compounds. Examples of these include gadolinium (Gd), samarium (Sm), neodymium (Nd), tungsten (W), tantalum (Ta), bismuth (Bi), hafnium (Hf), barium (Ba), dysprosium (Dy), and combinations thereof.

In an aspect, a disclosed therapeutic composition can be administered systemically to a subject. In an aspect, the subject can be a mammal. In an aspect, the mammal can be a primate. In an aspect, the mammal can be a human. In an aspect, the human can be a patient.

In an aspect, a disclosed therapeutic composition can be administered to a subject repeatedly. In an aspect, a disclosed therapeutic composition can be administered to the subject at least two times. In an aspect, a disclosed therapeutic composition can be administered to the subject two or more times. In an aspect, a disclosed therapeutic composition can be administered at routine or regular intervals. For example, in an aspect, a disclosed therapeutic composition can be administered to the subject one time per day, or two times per day, or three or more times per day. In an aspect, a disclosed therapeutic composition can be administered to the subject daily, or one time per week, or two times per week, or three or more times per week, etc. In an aspect, a disclosed therapeutic composition can be administered to the subject weekly, or every other week, or every third week, or every fourth week, etc. In an aspect, a disclosed therapeutic composition can be administered to the subject monthly, or every other month, or every third month, or every fourth month, etc. In an aspect, the repeated administration of a disclosed composition occurs over a pre-determined or definite duration of time. In an aspect, the repeated administration of a disclosed composition occurs over an indefinite period of time.

In an aspect, following the administration of a disclosed therapeutic composition, the cells are sensitized to treatment. In an aspect, following the administration of a disclosed therapeutic composition, a subject can be sensitized to treatment. In an aspect, an increased sensitivity or a reduced sensitivity to a treatment, such as a therapeutic treatment, can be measured according to one or more methods as known in the art for the particular treatment. In an aspect, the sensitivity of a cell or a subject to treatment can be measured or determined by comparing the sensitivity of a cell or a subject following administration of a disclosed therapeutic composition to the sensitivity of a cell or subject that has not been administered a disclosed therapeutic composition.

For example, in an aspect, following the administration of a disclosed therapeutic composition, the cell can be 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or greater, more sensitive to treatment than a cell that has not been administered a disclosed therapeutic composition. In an aspect, following the administration of a disclosed therapeutic composition, the cell can be 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, or greater, less resistant to treatment than a cell that has not been administered a disclosed therapeutic composition. The determination of a cell's or a subject's sensitivity or resistance can be routine in the art and within the skill of an ordinary clinician and/or researcher.

In an aspect, the determination of a cell's or a subject's sensitivity or resistance to treatment can be monitored. For example, in an aspect, data regarding sensitivity or resistance can be acquired periodically, such as every week, every other week, every month, every other month, every 3 months, 6 months, 9 months, or every year, every other year, every 5 years, every 10 years for the life of the subject, for example, a human subject or patient with traumatic brain injury. In an aspect, data regarding sensitivity or resistance can be acquired at various rather than at periodic times. In an aspect, treatment for a subject can be modified based on data regarding a cell's or a subject's sensitivity or resistance to treatment. For example, in an aspect, the treatment can modified by changing the dose of a disclosed compositions, the route of administration of a disclosed compositions, the frequency of administration of a disclosed composition, etc.

Disclosed herein is a therapeutic composition comprising at least one nanoparticle conjugated with a targeting ligand that binds to a biomarker of an injured, inflamed, diseased, or disrupted endothelial cell, wherein the nanoparticle further comprises one or more therapeutic agents encapsulated in the nanoparticle. The therapeutic composition is generally a drug for treating the injured, inflamed, diseased, or disrupted endothelial cell. In an aspect, the disclosed compositions or nanoparticles can comprise two or more therapeutic agents. Any combination of one or more drugs that can be encapsulated by the disclosed nanoparticles can be used. Examples include, but are not limited, a surfactant (like poloxamer surfactants), an antioxidant (like N-acetylcysteine).

In an aspect, the disclosed subject matter relates to pharmaceutical compositions comprising a disclosed composition comprising at least one nanoparticle conjugated with a targeting ligand that binds to injured, inflamed, diseased, or disrupted endothelial cells. In an aspect, the disclosed composition further comprises an imaging compound and one or more therapeutic agents encapsulated in the nanoparticle. In an aspect, the disclosed subject matter relates to pharmaceutical compositions comprising a therapeutic material for traumatic brain injury. In an aspect, a pharmaceutical composition can be provided comprising a therapeutically effective amount of at least one disclosed composition and a pharmaceutically acceptable carrier.

Methods of Making the Disclosed Composition

The drug-loaded nanoparticles can be produced using any suitable m ethos such as either by a) a high pressure homogenization-solvent evaporation technique or b) a double emulsion technique (water-in-oil-in-water emulsion). In some examples, the drug-loaded nanoparticles can be prepared by double emulsion technique. Typically, the polymer is dissolved in an organic solvent and the drug is dissolved in water. The aqueous solution is added to the organic phase. The mixture is emulsified to form a primary emulsion. The obtained water/oil emulsion is added to an aqueous solution of a stabilizing agent and then further emulsified. The stabilizing agents may include emulsifiers, surfactants or counterions, particularly polyvinyl alcohols, serum albumins, γ-cyclodextrin, and tocopheryl polyethylene glycol 1000 succinate (TPGS). The resulting coarse emulsion is passed through a high-pressure homogenizer. The homogenization step is repeated several times to produce a stable w/o/w emulsion. Then the organic solvent is removed by slow evaporation at ambient temperature and normal pressure.

The obtained nanosuspension is filtered through a glass-sintered filter. For storage, the resulting nanosuspension of drug-loaded nanoparticles can be freeze dried before they are coated with a targeting ligand. Preferably a cryoprotecting agent is added to the nanosuspension before it is freeze-dried. A suitable cryoprotecting agent is mannitol, which is preferably added to the nanosuspension in an amount of 5% (w/v).

Coating of the drug-loaded nanoparticles is preferably carried out with a solution of the targeting ligand in a solution and by allowing sufficient time to allow the targeting ligand to coat the drug-loaded nanoparticles.

It is possible to dissolve the drug and an additional emulsifier, such as γ-cyclodextrin, in water before adding the solution to the organic phase.

The obtained nanoparticle formulations are to be tested for resuspendability, particle size, drug loading (theoretical), and drug content.

It should be understood that the detailed description and specific examples, while indicating preferred embodiments of the invention, are given by way of illustration only, since various changes and modifications within the spirit and the scope of the invention will become apparent to those skilled in the art from this description and the accompanying figure as well as from the claims.

The drug targeting system for administering a pharmacologically active substance to the central nervous system of a mammal across its blood brain barrier, wherein the drug targeting system comprises nanoparticles made of poly(DL-lactide) and/or poly(DL-lactide-co-glycolide), a therapeutic agent, and a targeting ligand that binds endothelial cells. The drug targeting system is particularly suitable for administering pharmacologically active substances which have central nervous system activity but cannot cross the blood-brain barrier of a mammal without being modified or being associated with a carrier.

Methods of Using

Disclosed herein are methods for treating a subject with injured, inflamed, diseased, or disrupted endothelial cells by administering to the subject a composition as disclosed herein. in specific examples, the subject in need of treatment can be diagnosed with an acute traumatic brain injury or a chronic traumatic brain injury. In other examples, the subject may be diagnosed with diabetes or neuropathy. In certain examples, the disclosed methods can additionally comprise the administration of a third therapeutic agent, as part of a therapeutic regimen. The compounds can be delivered in the same dosage form or separately, and further can be taken concurrently or one subsequent to the other.

The composition may exhibit a burst release followed by a sustained release of one or more of the therapeutic agent. For example, the when the composition comprises a surfactant for repair of endothelial cells and an antioxidant, the composition exhibits an immediate burst release followed by a sustained release of the surfactant; and an immediate burst release of the antioxidant. The term “sustained release” is used in its conventional sense to refer to a drug formulation that provides for gradual release of a drug over an extended period of time, for example, 12 hours or more, and that preferably, although not necessarily, results in substantially constant blood levels of a drug over an extended time period. As used herein, the term “burst release” refers to a pharmaceutical preparation that provides for immediate release of a drug once the drug reaches the target, for example, within 3 hours or less, within 2 hours or less, or within 1 hour or less, and that preferably, although not necessarily, results in a substantial increase in blood levels of a drug over for a short time period.

Formulations

While it can be possible for nanoparticle composition to be administered as the neat nanoparticle composition, it is also possible to present them as a pharmaceutical formulation. Accordingly, provided herein are pharmaceutical formulations which comprise one or more of the disclosed nanoparticle composition together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients can be used as suitable and as understood in the art; e.g., in Remington: The Science and Practice of Pharmacy, 21^{st} Ed., Gennaro, Ed., Lippencott Williams & Wilkins (2003). The pharmaceutical compositions disclosed herein can be manufactured in any manner known in the art, e.g., by means of conventional mixing, dissolving, granulating, dragee-making, levigating, emulsifying, encapsulating, entrapping or compression processes.

A nanoparticle composition as provided herein can be incorporated into a variety of formulations for therapeutic administration, including solid, semi-solid, liquid or gaseous forms. The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route can depend upon for example the condition and disorder of the recipient. The formulations can conveniently be presented in unit dosage form and can be prepared by any of the methods well known in the art of pharmacy. Typically, these methods include the step of bringing into association a compound or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the nanoparticle composition disclosed herein suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient can also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders, inert diluents, or lubricating, surface active or dispersing agents. Molded tablets can be made by molding in a suitable machine a mixture of the powdered compound moistened with an inert liquid diluent. The tablets can optionally be coated or scored and can be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses. Also provided are oral formulations in the form of powders and granules containing one or more nanoparticle compositions disclosed herein.

The nanoparticle composition can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The formulations can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Formulations for parenteral administration include aqueous and non-aqueous (oily) sterile injection solutions of the nanoparticle composition which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. Optionally, the suspension can also contain suitable stabilizers to allow for the preparation of highly concentrated solutions.

In addition to the formulations described previously, the nanoparticle composition can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions can take the form of tablets, lozenges, pastilles, or gels formulated in conventional manner. Such compositions can comprise the active ingredient in a flavored basis such as sucrose and acacia or tragacanth.

The nanoparticle composition can also be formulated in rectal compositions such as suppositories or retention enemas, e.g., containing conventional suppository bases such as cocoa butter, polyethylene glycol, or other glycerides.

For administration by inhalation, nanoparticle composition can be conveniently delivered from an insufflator, nebulizer pressurized packs or other convenient means of delivering an aerosol spray. Pressurized packs can comprise a suitable propellant such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Alternatively, for administration by inhalation or insufflation, the compounds can take the form of a dry powder composition, for example a powder mix of the compound and a suitable powder base such as lactose or starch. The powder composition can be presented in unit dosage form, in for example, capsules, cartridges, gelatin or blister packs from which the powder can be administered with the aid of an inhalator or insufflator.

In one example, a compound is prepared for delivery in a sustained-release, controlled release, extended-release, timed-release or delayed-release formulation, for example, in semipermeable matrices of solid hydrophobic polymers containing the therapeutic agent. Various types of sustained-release materials have been established and are well known by those skilled in the art. Current extended-release formulations include film-coated tablets, multiparticulate or pellet systems, matrix technologies using hydrophilic or lipophilic materials and wax-based tablets with pore-forming excipients (see, for example, Huang, et al. Drug Dev. Ind. Pharm. 29:79 (2003); Pearnchob, et al. Drug Dev. Ind. Pharm. 29:925 (2003); Maggi, et al. Eur. J. Pharm. Biopharm. 55:99 (2003); Khanvilkar, et al., Drug Dev. Ind. Pharm. 228:601 (2002); and Schmidt, et al., Int. J. Pharm. 216:9 (2001)). Sustained-release delivery systems can, depending on their design, release the compounds over the course of hours or days, for instance, over 4, 6, 8, 10, 12, 16, 20, 24 hours or more. Usually, sustained release formulations can be prepared using naturally-occurring or synthetic polymers, for instance, polymeric vinyl pyrrolidones, such as polyvinyl pyrrolidone (PVP); carboxyvinyl hydrophilic polymers; hydrophobic and/or hydrophilic hydrocolloids, such as methylcellulose, ethylcellulose, hydroxypropylcellulose, and hydroxypropylmethylcellulose; and carboxypolymethylene.

The sustained or extended-release formulations can also be prepared using natural ingredients, such as minerals, including titanium dioxide, silicon dioxide, zinc oxide, and clay (see, U.S. Pat. No. 6,638,521, herein incorporated by reference). Exemplified extended release formulations that can be used in delivering a compound include those described in U.S. Pat. Nos. 6,635,680; 6,624,200; 6,613,361; 6,613,358, 6,596,308; 6,589,563; 6,562,375; 6,548,084; 6,541,020; 6,537,579; 6,528,080 and 6,524,621, each of which is hereby incorporated herein by reference. Controlled release formulations of particular interest include those described in U.S. Pat. Nos. 6,607,751; 6,599,529; 6,569,463; 6,565,883; 6,482,440; 6,403,597; 6,319,919; 6,150,354; 6,080,736; 5,672,356; 5,472,704; 5,445,829; 5,312,817 and 5,296,483, each of which is hereby incorporated herein by reference. Those skilled in the art will readily recognize other applicable sustained release formulations.

Systemic administration can also be by transmucosal or transdermal means. For transmucosal or transdermal administration, penetrants appropriate to the barrier to be permeated are used in the formulation. For topical administration, the agents can be formulated into ointments, creams, salves, powders or gels. In one embodiment, the transdermal delivery agent can be DMSO. Transdermal delivery systems can include, e.g., patches. For transmucosal administration, penetrants appropriate to the barrier to be permeated are used in the formulation. Such penetrants are generally known in the art. Exemplified transdermal delivery formulations that can find use with the compounds disclosed herein include those described in U.S. Pat. Nos. 6,589,549; 6,544,548; 6,517,864; 6,512,010; 6,465,006; 6,379,696; 6,312,717 and 6,310,177, each of which are hereby incorporated herein by reference.

The precise amount of nanoparticle composition administered to an individual will be the responsibility of the attendant physician. The specific dose level for any particular individual will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration can vary depending on the condition and its severity. The dosage can be increased or decreased over time, as required by an individual. An individual initially can be given a low dose, which is then increased to an efficacious dosage tolerable to the individual. Typically, a useful dosage for adults can be from 5 to 2000 mg, but have been known to range from 0.1 to 500 mg/kg per day. By way of example, a dose can range from 1 to 200 mg, when administered by oral route; or from 0.1 to 100 mg or, in certain embodiments, 1 to 30 mg, when administered by intravenous route; in each case administered, for example, from 1 to 4 times per day. When a compound is administered in combination with another therapeutic agent, a useful dosage of the combination partner can be from 20% to 100% of the normally recommended dose, since, as discussed below, even doses of a given drug which would be subtherapeutic if administered on its own can be therapeutic when used in combination with another agent.

Dosage amount and interval can be adjusted individually to provide plasma levels of the active compounds that are sufficient to maintain therapeutic effect. In certain examples, therapeutically effective serum levels will be achieved by administering single daily doses, but efficacious multiple daily dose schedules can be used as well. In cases of local administration or selective uptake, the effective local concentration of the drug cannot be related to plasma concentration. The skilled practitioner will be able to optimize therapeutically effective local dosages without undue experimentation. Additionally, applicable methods for determining an appropriate dose and dosing schedule for administration of compounds such as those disclosed herein are described, for example, in Goodman and Gilman's The Pharmacological Basis of Therapeutics, 11^{th} Ed., Brunton, Lazo and Parker, Eds., McGraw-Hill (2006), and in Remington: The Science and Practice of Pharmacy, 21^{st} Ed., Gennaro, Ed., Lippencott Williams & Wilkins (2003), both of which are hereby incorporated herein by reference.

In certain instances, it can be appropriate to administer at least one of the nanoparticle composition described herein (or a pharmaceutically acceptable salt, ester, or prodrug thereof) in combination with a third therapeutic agent.

Numerous characteristics and advantages provided by aspects of the present disclosure have been set forth in the foregoing description and are set forth in the attached Appendix, together with details of structure and function. While the present disclosure is disclosed in several forms, it will be apparent to those skilled in the art that many modifications can be made therein without departing from the spirit and scope of the present disclosure and its equivalents. Therefore, other modifications or embodiments as may be suggested by the teachings herein are particularly reserved.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Engineering Delivery of Non-Biologics Using PLGA Nanoparticles for Repair of Disrupted Brain Endothelium

ABSTRACT: Traumatic brain injury (TBI) is known to alter the structure and function of blood-brain barrier (BBB). Blunt force or explosive blast impacting the brain can cause neurological sequelae through the mechanisms that remain yet to be fully elucidated. For example, shockwaves propagating through the brain have been shown to create a mechanical trauma that may disrupt the BBB. Indeed, using tissue engineering approaches, the shockwave-induced mechanical injury has been shown to modulate the organization and permeability of the endothelium tight junctions. Because an injury to the brain endothelium typically induces a high expression of E-selectin, we postulated that up-regulation of this protein following an injury can be exploited for diagnosis and potential therapy through targeted nano-delivery to the injured brain endothelium. To test this hypothesis, we engineered poly (lactic-co-glycolic acid; PLGA) nanoparticles to encapsulate therapeutic non-biologics and decorated them with ligands to specifically target the E-selectin. A high level of the conjugated nanoparticles was found inside the injured cells. Repair of the injury site was then quantitatively measured and analyzed. To summarize, exploiting the tunable properties of PLGA, a targeted drug delivery strategy has been developed and validated that combines the specificity of ligand/receptor interaction with therapeutic reagents. Such a strategy could be used to provide a potential theragnostic approach for treatment of modulated brain endothelium associated with TBI.

INTRODUCTION: Nanoparticles (NPs) can function as both diagnostic and therapeutic agents (i.e., theragnostic) and may be used as tools for delivering therapeutic drugs to the specific targets, hence minimizing systemic administration of drugs and potential toxicity. In addition, biocompatible and conjugated NPs may avoid being removed from the circulation, and thereby enhance the number of NPs that can reach the intended target. Various pharmaceutical nanotechnology-based systems like liposomes, carbon nanotubes, quantum dots, dendrimers, polymeric nanoparticles, metallic nanoparticles, and others have brought about revolutionary changes in drug delivery. Nano-sized objects can be transformed in numerous ways to alter their characteristics. Active targeting by modifying the NP surface with peptides or antibodies has been shown to improve the cell-specificity.

In the present example, PLGA NPs were fabricated, loaded with drugs, and conjugated with P-selectin glycoprotein ligand-1 (PSGL-1) to specifically target the injured tissue and induce repair and restore the tissue's structural and functional integrity. PSGL-1 is a glycoprotein found on white blood cells and endothelial cells. PSGL-1 can bind to all three members of the family (P-selectin, E-selectin, and L-selectin) that is part of the broader family of cell adhesion molecules. The P-selectin expression peaks relatively early after activation (˜10 min), while the E-selectin expression continue to increase even after 6 hours. Therefore, conjugating NPs with PSGL-1 is expected to lead to an early phase binding of the NPs to the cells via P-selectin, followed by more sustained binding to E-selectin.

Experimental results herein show the cell adhesion molecules (e.g., E-selectin) are up-regulated in response to shockwave-induced lesions in the tissue engineered brain endothelium model. The injured brain endothelium can be repaired by delivering PLGA NPs specifically designed to bind to E-selectin on the injured endothelial cells, internalized and release the contents of two non-biologics for structural and functional restoration. The surfactant poloxamer 188 (P188) and antioxidant N-acetylcysteine (NAC), both FDA-approved, were encapsulated into the NPs to repair the injured endothelial cells. It appears evident that the NPs conjugated with specific ligand and loaded with P188 and NAC can target, diagnose, deliver therapeutic drugs, and enhance restoration of the brain endothelium. Moreover, the biosystem developed for this example validate the efficacy of nano-delivery to repair the brain endothelium as well as the ability to engineer and reproduce. It may provide an inexpensive platform to rapidly screen potential theragnostic nano-delivery of drugs and reagents to restore the brain endothelium.

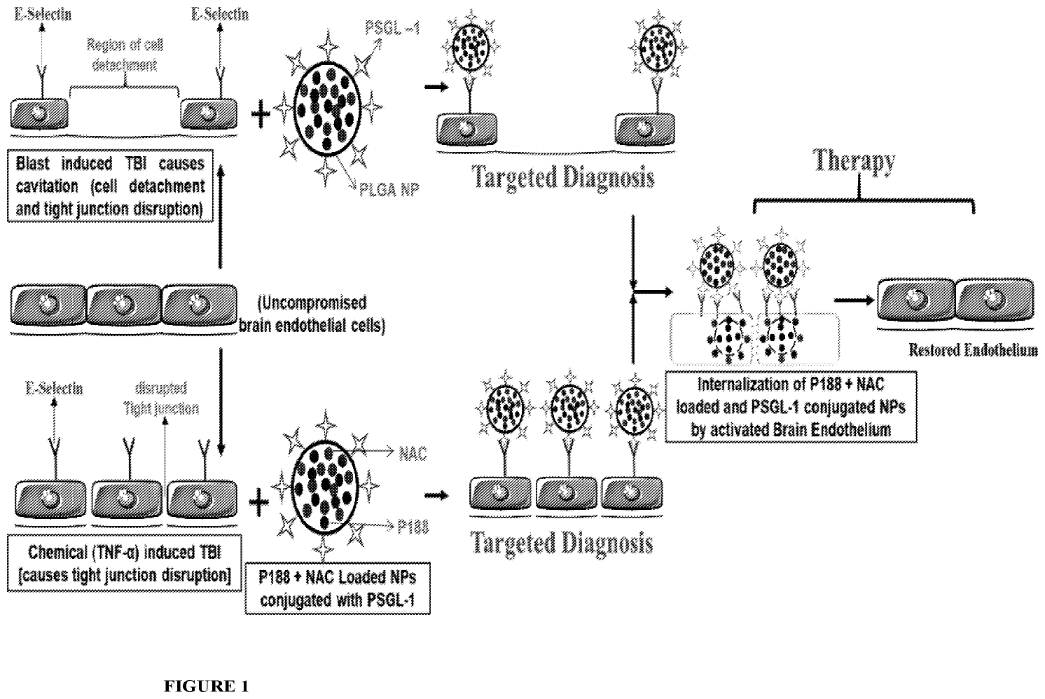

RESULTS AND DISCUSSION: Overall experimental strategy and aims of the current example are outlined in FIG. 1. An in vitro brain endothelium model was developed to create shockwave-induced, localized sub-mm size lesion in which cells were detached by the mechanical trauma. The injured endothelial cells around the periphery of the lesion were first identified and then targeted by engineering NPs. The PLGA NPs are decorated with specific ligands to E-selectin and encapsulated with at least two non-biologic compounds (P188 and NAC) that have been proven to demonstrate reparative effects. The extent of endothelium repair was quantified by examining closure of the lesion, at least partial reformation of the tight junctions, and then measuring the permeability of tracer molecules (10 kDa) through the brain endothelium model to test the biotransport functionality.

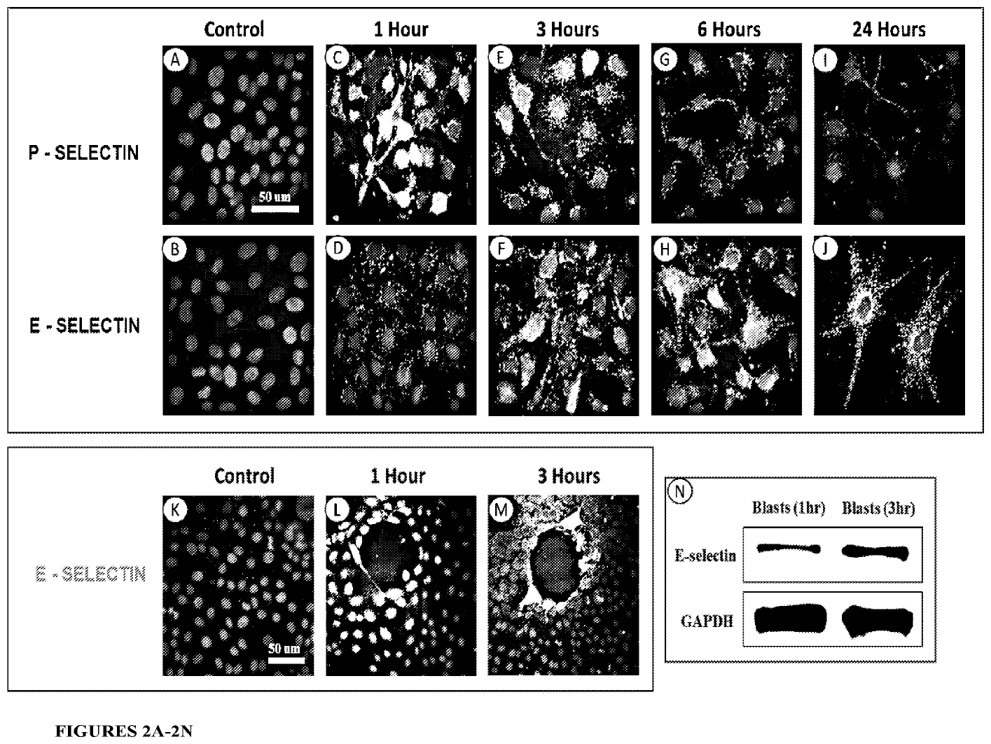

E-Selectin Expression. Targeting injured inflammatory endothelial cells was probed by validating up-regulation of E-selectin. Brain endothelial cells (BECs) were incubated with an inflammatory factor (TNF-α) to generate inflammatory responses and used it as a model for disruption of the brain endothelium. The rationale was two-fold. First, a trauma was generated using a powerful cytokine and its effects on the brain endothelium can then be determined and compared to those induced by a mechanical trauma (microcavitation). Second, suitable cell surface molecules would have to be identified for targeting and delivery of some compounds with potential reparative capability. Following a treatment of BECs with TNF-a (10 ng/ml) for 4 hours, time-dependent expressions of both E- and P-selectin were monitored, because the selectins are expressed preferentially in the injured, inflamed, diseased, or disrupted endothelial cells. It was observed that a high expression of P-selectin within an hour after the TNF-a administration, but E-selectin expression was not readily evident (FIG. 2C). After 3 hours, higher expressions of both E- and P-selectin were detected (FIGS. 2E and 2F). However, after 6 hours, the expression of P-selectin was visibly diminished (FIG. 2G), but the expression of E-selectin still remained elevated (FIG. 2H) and persisted up to 24 hours (FIG. 2J). Based on these results, the E-selectin appears as a more suitable candidate and therefore was chosen as a molecular target of injured endothelial cells. Because therapeutic interventions for brain trauma are typically administered at later stages, it may be clinically more relevant to focus on the markers that may be detected at earlier stages but are sustained until later stages. The expression of E-selectin appears to satisfy these criteria, providing feasibility to targeted delivery of NPs to the injured endothelial cells. Next, the E-selectin expression was then examined in response to a mechanical trauma (microcavitation). One of the characteristics of this particular mechanical injury was shown to create a lesion in which the cells are detached following the collapse of highly pressurized microbubbles. This mechanically induced event is explicitly demonstrated by formation of approximately a circular area (˜100 mm diameter; FIG. 2L) in which endothelial cells are detached. There are two observations to note. First, blood cells, toxins and large molecules from the circulating blood can easily diffuse through the lesion and could be responsible for the sequelae associated with blast TBI. Second, the endothelial cells around the periphery of the lesion were still attached to the substrate and showed a high expression of E-selectin that remained noticeably up-regulated after 3 hours following the mechanical trauma (FIG. 2M). The fluorescent image results were confirmed by Western blot analysis that showed an up-regulation of E-selectin (FIG. 2N). These results were important to validate that E-selectin is a viable target for nano-delivery to potentially treat the mechanically-induced lesion.

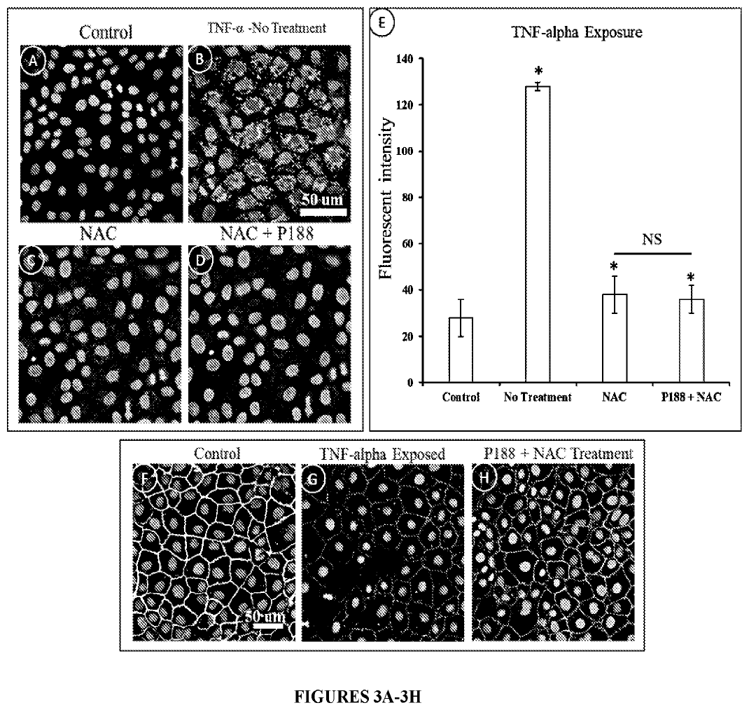

Next tested was the potential of reparative effects of two non-biologics. One is a triblock biocompatible polymer, known as poloxamers (P188), that was approved by FDA for numerous applications. Because of the structural resemblance of P188 to the phospholipid bilayer, it has been shown to reseal the damaged cell membrane and facilitate repair of injured endothelial cells. The other compound is N-acetylcysteine (NAC), which is also an FDA-approved antioxidant that is associated with health benefits. Both P188 and NAC are non-biologic and have been shown to attenuate the potential harmful effects of oxidative stress. In order to demonstrate and establish the effects of P188 and NAC in response to oxidative stress, BECs were stimulated by TNF-a (10 ng/ml) for 4 hours and then treated with NAC or the combination of P188+NAC. Using a fluorophore (MitoSox) that preferentially binds to superoxide, the extent of oxidative stress in response to TNF-a were recorded and quantified. Oxidative stress is an important determinant of endothelial injury, which influences a number of cellular responses by activating several intracellular signaling cascades in endothelial cells, leading to the progression of vascular diseases. As illustrated in FIG. 3, there was essentially no superoxide production in control cells (FIG. 3A). TNF-a induced a significant increase in oxidative stress and, if left untreated, the superoxide level persisted (FIG. 3B). Treatment of the cells with either NAC alone (FIG. 3C) or the combination of NAC+P188 (FIG. 3D) showed a noticeable attenuation of superoxide. As expected, NAC was found to more effective than P188. More interestingly, when the combination of P188+NAC was applied, oxidative stress within the cells was diminished only negligibly (FIG. 3E). This result suggests that, although P188 can also reduce the ROS levels, an antioxidant would act faster to suppress the oxidative stress, and then reparative effects of P188 are expected to follow (shown below). This sequence of events may be engineered to accommodate short- and long-term beneficial responses. Another important role of brain endothelial cells is to regulate biotransport properties across the blood brain barrier. The mouse primary brain endothelial cells used in this study were characterized by visualizing tight junction proteins (ZO-1). Tight junctions in control cells are clearly demonstrated, showing tightly packed ZO-1 proteins (FIG. 3F) and a well-defined monolayer of BECs. When the cells were exposed to TNF-α, the tight junction proteins (ZO-1) diminished considerably and disorganized (FIG. 3G). However, when the cells were treated with the NAC+P188 cocktail, the tight junctions appeared to have been partially but noticeably restored (FIG. 3H). This is consistent with restoration and repair of tight junctions by P188 in response to a mechanical trauma and recovery of the permeability through the brain endothelium. Disrupted tight junctions in response to inflammatory insult may be reversed by treating the cells with the combination of NAC+P188.

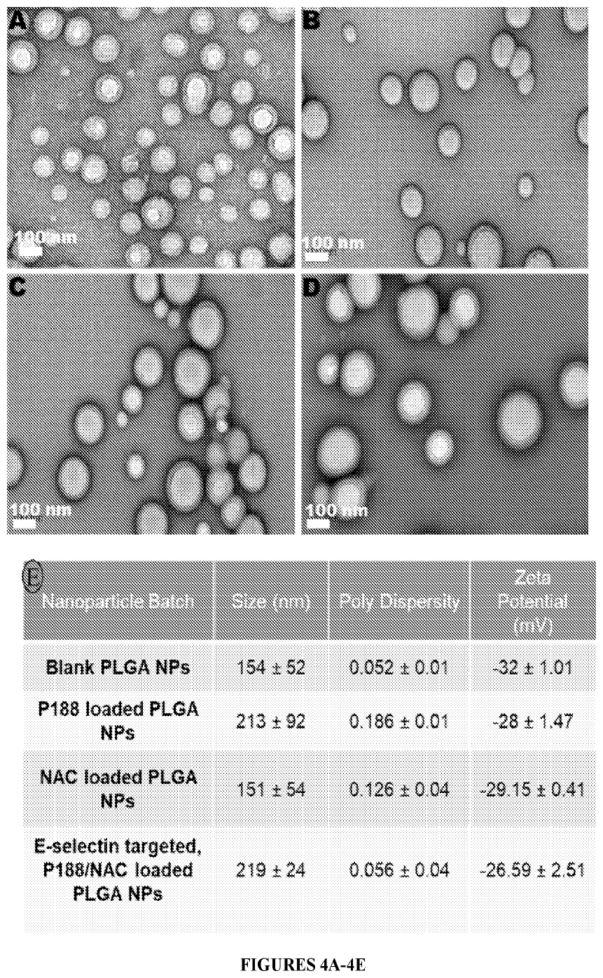

Having validated the effects of P188 and NAC on the attenuation of oxidative stress and restoration of tight junctions, the next challenge was to encapsulate them into nanoparticles for targeted delivery to injured endothelial cells only. A double emulsion method was used to generate poly (lactic-co-glycolic acid) (PLGA) nanoparticles. PLGA has been used in a host of FDA-approved therapeutic devices with proven biocompatibility and extensively studied as delivery vehicles for DNA, drugs, proteins, and peptides. Also, the physical properties of the polymer can be tuned by controlling the molecular weight and the ratio of lactide to glycolic acid. Its concentration can be manipulated to achieve the desired dosage and release interval. Fabricated PLGA NPs were characterized under 4 different conditions using transmission electron microscopy (TEM) and dynamic light scattering. To prepare the NPs for this study, PLGA-50-50 (24,000-30,000 MW) was used for fabrication. TEM images indicated that all NPs maintained uniform size (FIG. 4A to 4D) and the zeta potential (FIG. 4E). The loading efficiency for P188 and NAC was also determined to be 85% and 28% by dissolving the NPs and measuring the contents for estimation of loading efficiency. The low loading efficiency of NAC, when compared to P188, is attributed to factors such as the molecular weight of NAC (˜40× less than that of P188), which is expected to cause rapid diffusion from the nanoparticles.

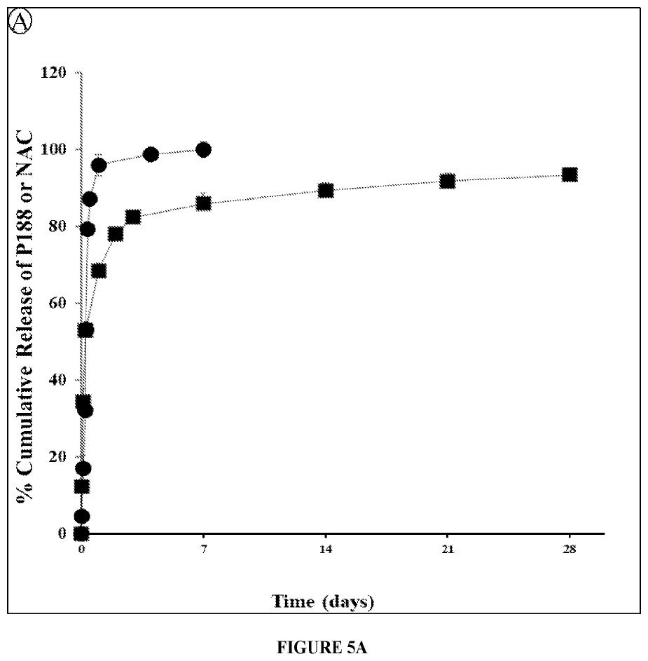

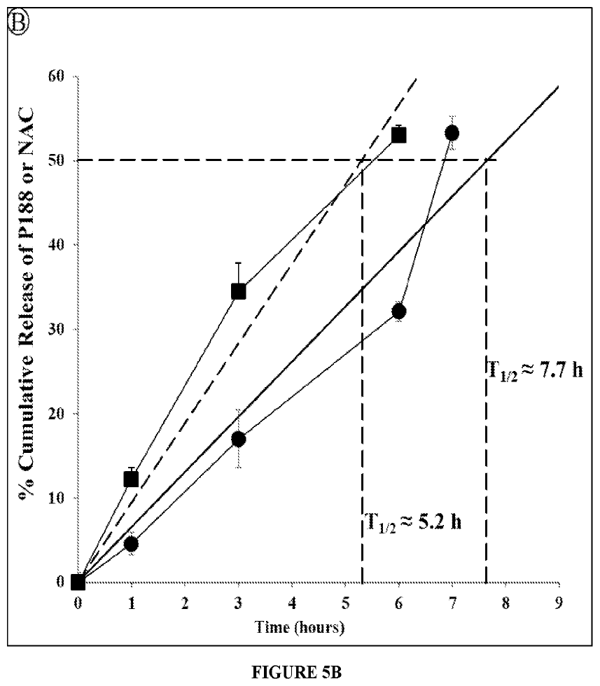

Release kinetics of P188 and NAC from PLGA NPs were next determined. The amount of drug loaded in the delivery particle plays an important role in the rate and duration of drug release. It is speculated that particles with a higher drug content possess a larger initial burst release than those having lower content because of their smaller polymer to drug ratio. Hydrophobic interactions and rapid degradation of particles have also been shown to play a role in the burst release. For P188, most of the release took place after day 3 and then sustained from day 7 until day 28 (FIG. 5). NAC release was rapid as most of its release was achieved within 24 hours and approached near 100% release in 7 days, and hence no additional measurements were performed. The faster release rate of NAC can be attributed to its molecular weight and the porosity of NPs. However, the rapid release of NAC may be essential for the aims of this example, because it can induce early attenuation of ROS before the onset of P188 therapeutic potential that might include promoting cell migration and proliferation, and therefore spanning different time scales to trigger reparative machineries inside the cell. A linear regression analysis showed that the time required to reach a half of the maximum release for NAC was ˜7.7 hours, whereas that for P188 was ˜5 hours. Although P188 has larger molecular weight, the loading efficiency was greater than that of NAC (85% vs. 28%). Thus, the initial burst release of P188 was not unexpected.

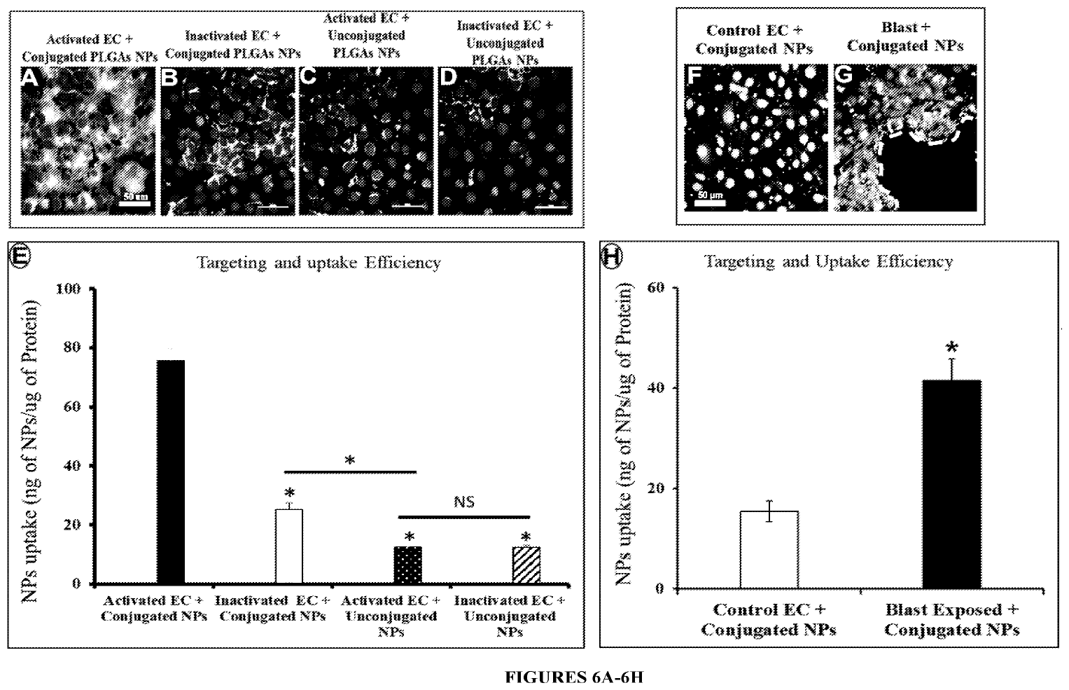

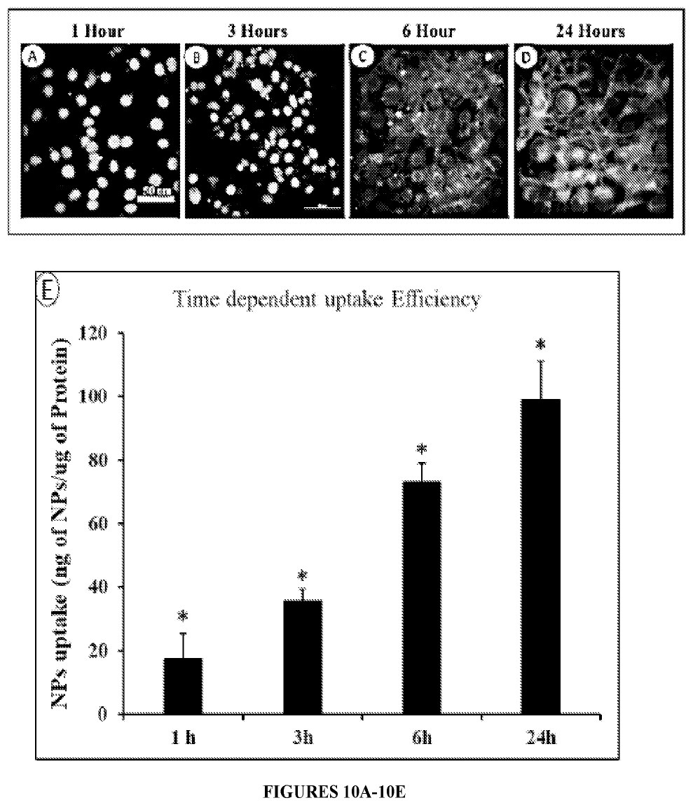

Conjugation, binding, and cellular uptake. To validate conjugation and cellular uptake of conjugated NPs, BECs were incubated with NPs that were conjugated with PSGL-1 (specific ligand against E-selectin). The conjugation efficiency was calculated and determined to be 41.86±0.13%. The binding and internalization of PSGL-1 conjugated PLGA NPs into TNF-α activated brain endothelial cells were next determined. Fluorescently labeled (FITC) PSGL-1 conjugated PLGA NPs were internalized by activated endothelial cells (FIG. 6A), whereas TNF-α activated cells but incubated with unconjugated PLGA NPs had a much diminished, detectable internalization (FIG. 6B). To determine whether the resulting conjugated FITC-PLGA NPs retain the target specificity to the E-selectin-expressing cells, non-targeting FITC-PLGA-NPs were incubated with activated and inactivated endothelial cells for 6 hours and a very small number of FITC-PLGA-NPs were detected in the endothelial cells (FIGS. 6C and 6D). Protein concentrations were used to normalize and quantify the internalization of NPs (FIG. 6E). A time-dependent study was carried out next to determine the duration of time needed for most NPs to be internalized. The results showed that the longer the incubation time, the higher the number of NPs internalized, as expected. Nearly 80% of the conjugated NPs were internalized after 6 hours, and it approached 100% internalization following a 24-hour incubation (FIG. 10). Reparative effect of the conjugated P188+NAC NPs in response to mechanically induced lesion was next tested. In control cells that were not exposed to microcavitation, only a small number of NPs was visualized (FIG. 6F). When exposed to mechanical trauma, the cells at the periphery of the cell detachment area showed an increased level of NPs in these cells, suggesting an abundance of internalized (P188+NAC)-encapsulated NPs (FIG. 6H). This represents a set of confirmatory experiments that again highlight the specificity of NPs targeting the injured endothelial cells.

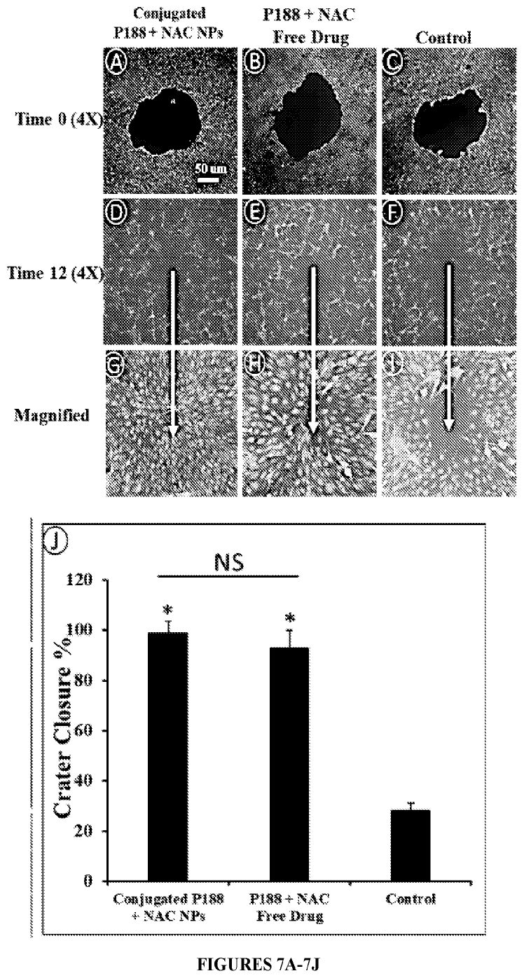

The important question was whether NPs loaded with the two non-biologics can repair the lesion. Monolayers of BECs were pre-incubated with a green cell tracker and exposed to the mechanical trauma. Again, the in vitro wound was visibly recognizable (FIG. 7A to 7C). These cells were (1) left untreated (control); (2) treated with the conjugated P188+NAC NPs for 12 hours; or (3) treated with P188+NAC free drug for 12 hours without using NPs (positive control). Fluorescent images were then recorded (FIG. 7D to 7F) and compared to those recorded at the initial time point. To accentuate the effects, images were magnified and the in vitro wound closure was determined. Image analyses indicate that a 99% closure of the lesion was achieved by applying the conjugated P188+NAC NPs, as compared to a 93% closure using free drug delivery method (FIG. 7J); however, there was no statistically significant difference. If left untreated, the wound closure was only 28%. This provides concrete evidence that repair of a mechanically induced sub-mm lesion is plausible by delivering decorated NPs to the injured endothelial cells. While NAC was fast-acting to suppress oxidative stress (see FIG. 3), P188 can promote and enhance cell proliferation and migration into the lesion. It should be noted that P188 is not absorbed by the gut and typically delivered intravenously. Two alternative delivery mechanisms can now be contemplated. First, NPs may be delivered via intranasal pathway, which can bypass the blood-brain barrier. Second, because the goal of this study is to target the injured endothelial cells, a non-invasive delivery of NPs containing NAC and P188 may be envisioned through formulation of indigestible capsules for easy administration and storage.

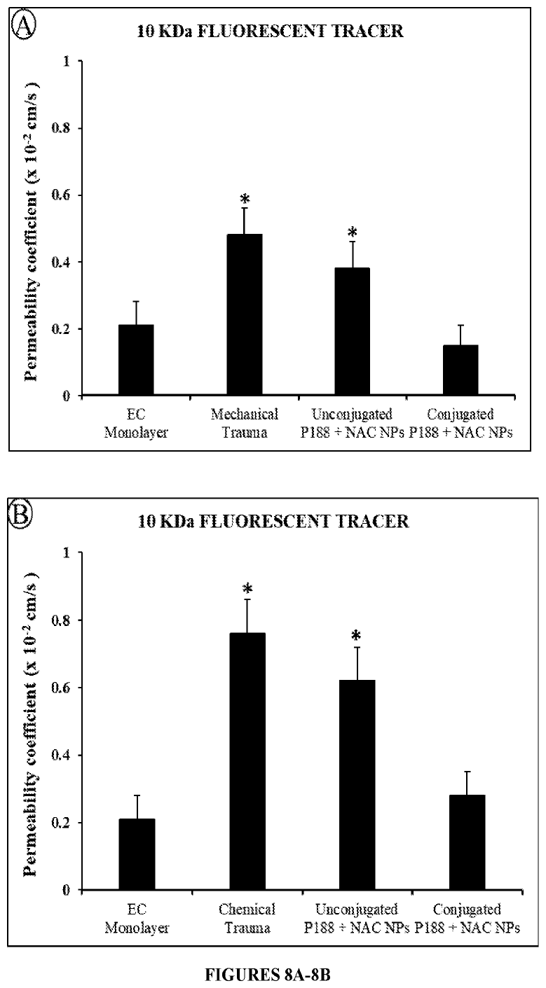

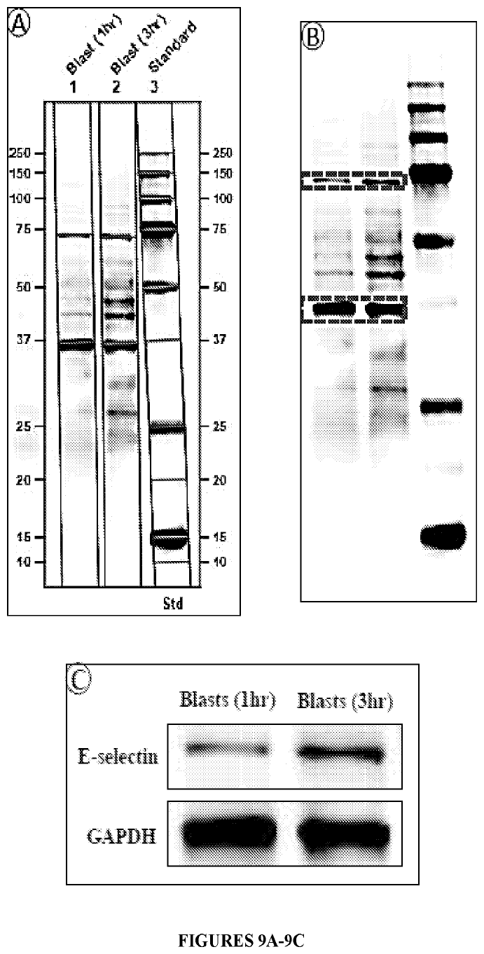

Therapeutic effect of conjugated P188+NAC NPs on BECs permeability. The therapeutic potentials of conjugated P188+NAC NPs were further validated by measuring the permeability of large molecules (e.g., 10 kDa Dextran) across the brain endothelium model exposed either to TNF-α or mechanical microcavitation. The diffusion chamber was custom-designed and engineered for permeability studies and has been described in detail elsewhere. The rationale was that, since the mechanical trauma creates a lesion of ˜100 mm in diameter, toxins and blood cells from circulation can easily diffuse through the lesion and adversely affect the brain tissue. Since 10 kDa tracer molecules are relatively large, the permeability should be negligible in the control brain endothelium (FIG. 8). Interestingly, either the mechanical trauma or the TNF-a cytokine increased the permeability significantly by several folds. This apparent leaky endothelium was restored by the treatment of NAC+P188 NPs for 12 hours. This provides yet another biophysical evidence that the conjugated and encapsulated NPs restore the functionality of the brain endothelium in addition to the demonstrated structural restoration (see FIG. 3). Collectively, our results validate the diagnostic and therapeutic potential of administrating conjugated P188+NAC NPs to the brain endothelium and repair of the disrupted blood-brain barrier.

CONCLUSION: The development of targeted therapeutic approaches that have been demonstrated in the current study provide a means to address an unmet clinical challenge for the treatment of traumatic brain injuries (see FIG. 1). This example validated the use of fluorescent NPs to visualize internalization in activated endothelial cells in response to a cytokine or mechanical trauma. It is reported for the first time that a novel combination of P188+NAC has efficacious effects on the repair of brain endothelium. The combination delivery to the injured cells appears to promote cell proliferation and migration into a lesion and reestablished the structural and functional integrity of the brain endothelium. Taken together, these results establish a platform for the development of a novel theragnostic treatment to regenerate the disrupted brain endothelium using non-biologics. The platform is also expected to contribute to additional development and validation of therapies for brain pathologies.

Experimental Section

Preparation of P188, NAC, and P188-NAC loaded PLGA NPs. Drugs loaded to PLGA NPs by standard double emulsion method with minor modifications. Briefly, the drug solution (water phase-w1) was formed by adding (either 10 mg of P188 or 10 mg of NAC or combination dissolved 100 ul of DI water) and was emulsified into 2.5% (w/v) PLGA solution prepared in 1.75 ml of chloroform and sonicated (˜30 s to 1 min at 10 to 20 W). This primary emulsion was added drop wise into 12 ml of 5% (w/v) PVA (85-88% hydrolyzed, 13 kDa) and sonicated again at 30 W for 5 min. following the overnight stirring to evaporate organic solvents, the nanoparticles were washed and isolated by centrifugation at 15,000 rpm for 30 min, and P188+NAC-loaded NPs were collected via freeze-drying.

NPs size, Zeta Potential, and Morphology analysis. One mg of each prepared NPs was suspended in DI water, mixed, and measured. The mean size, size distribution, and zeta potential of prepared NPs were measured by dynamic light scattering using ZetaPALS zeta potential analyzer (Brookhaven Instruments, Holtsville, N.Y.). The average effective diameter of the nanoparticles was reported. The data were averages of three measurements. The morphology of the NPs was examined by TEM. Briefly, particles were dropped onto a carbon-coated-on lacey support film and allowed to dry before characterization.

Drug Encapsulation Efficiency. To find the amount of P188 and NAC within the NPs, P188 was fluorescently modified (TAMRA-P188) and loaded into the NPs. Fluorescence intensity of TAMRA-P188 in the supernatant was quantified over time using a plate reader. NAC was measured with high-performance liquid chromatography (HPLC). The results were converted into concentrations, and the loading efficiency for P188 and NAC were 85% and 28%, respectively, and calculated using the following equation.

$$Loading Efficiency = \frac{Original Drug Amount - Supernatant Drug Amount}{Original Drug Amount} * 100 \qquad ( 1 )$$

In vitro Drug Release. For the release studies, 1 mg each from all prepared NPs were dispersed in 1 ml of release medium (pH 7.4) each to form a suspension. Each suspension was in three replicates in 1.5 ml centrifuge tubes and incubated at 37° C. The release buffers were collected after centrifugation at 15,000 rpm for 30 min. The release buffers were replaced with fresh buffer at different periods and subjected to analysis using HPLC. The cumulative release of the model drug from each loaded NPs was plotted against time.

Cell Culture Technique and Characterization. BALB/c Mouse Primary Brain Microvascular Endothelial Cells (MPBMECs, Cell Biologics) were grown in Endothelial Basal Medium-2 (EBM-2) with EGM-2 kit (Lonza) in a flask coated with a gelatin-based coating solution. MPBMECs (6.6×10⁴ cells/cm²) were directly seeded unto the cover glass coated with fibronectin (1 μl/mL) and cultured with EBM-2. Confluent cells were characterized by the formation of tight junction.

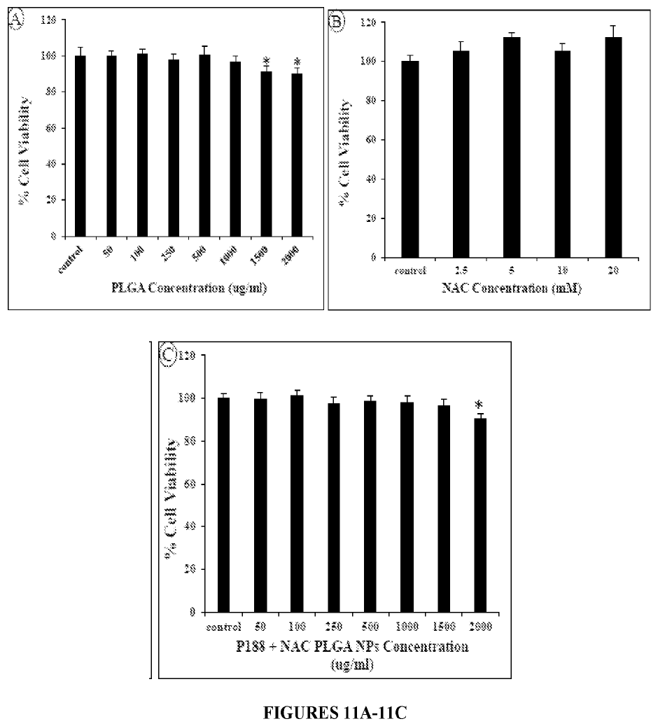

Brain Endothelial Cell Viability. Viability of cells following a 24-hour exposure to blank PLGA NPs (blank NPs), P188, NAC, and P188+NAC NPs was assessed with the MTT assay. It measures the metabolic conversion of the MTT salt (3-[4,5-dimethylthiazol-2-yl]-2,5-diphenyltetrazolinum bromide) by active mitochondrial dehydrogenases. Briefly, after exposure of BECs to blank NPs, P188, NAC, and P188+NAC NPs, cells were treated with MTT (5 mg/ml) for 1 hour at 37° C. The formazan product generated was solubilized by the addition of 20% sodium dodecyl sulfate and 50% N, N-dimethylformamide, and quantified by measuring its absorbance at 490 nm. Untreated cells were taken as control with 100% viability. Resulting sample absorbance was used to calculate cell viability. The results of cell viability experiments were analyzed and shown in FIG. 11.

Endothelial Cells Activation. Confluent cells were exposed to TNF-α (10 ng/ml) for 1 to 24 hours and then stained for protein expression. Images were acquired to investigate the E- and P-Selectin expression after 1, 3, 6, and 24 hours. This period was chosen based on immunofluorescence observations that the earliest expression of inflammatory proteins on activated endothelial cells occurs between 1 to 6 hours, and the sustained expression of the selectins after 24 hours was of interest to complete this example.

Immunostaining for E & P-Selectin, and PSGL-1. Inflammatory markers in the brain endothelial cells were visualized using E & P-Selectin antibodies. Briefly, cells were activated by mechanical (shockwave) and cytokine (TNF-α) induction, fixed with 4% paraformaldehyde in PBS, permeabilized with 0.1% Triton X-100 for 3 min and blocked with 3% of BSA for 1 hour. Cells were incubated with the E- or P-selectin primary antibodies (1:500, 4.3 μg/ml, Santa Cruz, SC137054 and SC271267, respectively), and subsequently incubated with Alexa Fluor 488-conjugated goat anti-mouse IgG (1:1000, 2 μg/ml, Santa Cruz, SC516167). Nuclei were counter-stained with DAPI.

Measurement of Mitochondrial Reactive Oxygen Species (ROS). Superoxide was measured using a MitoSoX™ Red mitochondrial superoxide indicator, according to the manufacturer's instructions. Briefly, BECs were cultured on glass cover slides and exposed to TNF-α for 4 hours before treating with P188, NAC, or P188+NAC for 12 hours. After treatments, the cells were incubated with 1 μM MitoSoX™ reagent working solution for 30 min at room temperature. After washing three times with PBS, images were taken with a fluorescent microscope and fluorescence intensities were calculated using Nikon ND2 software.

Conjugation of PSGL-1 to PLGA NPs. The functional ligand PSGL-1 was bound to the surface of the PLGA NPs via the EDC-NHS chemistry with modification. Briefly, 120 mg of EDC added to 180 mg NHS and dissolved in 5 ml of MES buffer (0.1 M, pH 4.75). 20 mg PLGA NPs were resuspended in the EDC-NHS solution and rotated for 2 hours at room temperature. After 2 hours of incubation at room temperature with rotation, the resulting NPs were ultracentrifuge at 15,000 rpm for 30 min at 4° C., washed three times with PBS, and then resuspended in PBS (1 mg/ml). 80 ul of 500 ug/ml of mouse PSGL-1 antibody (Sino Biological, 50770-MCCH) was added to the NPs solution and incubated for 24 hours at 4° C. Following the incubation, the NPs were collected by ultracentrifugation at 15,000 rpm for 30 min. The supernatant was used to determine the peptide conjugation efficiency. Pellets were resuspended in DI water, freeze-dried, and stored for use. The supernatant was quantified using bicinchoninic acid (BCA) protein assays following manufacturer's instructions, and unconjugated peptides (PSGL-1) was analyzed based on bovine serum albumin (BSA) standards.

Cellular Internalization of Conjugated FITC labeled PLGA NPs. Cells (2×10⁴ cells per well) were cultured in a 96 well plate in Dulbecco's Modified Eagle's Medium (DMEM) supplemented with 2% fetal bovine serum (FBS) and 1% of penicillin at 37° C. for 24 hours. FITC-loaded NPs (1 mg/ml) was used for the observation of cellular internalization at 37° C. for 6 hours. Conjugated FITC NPs were added to two columns of the 96 well plate (one column activated with TNF-α and the other column inactivated), and unconjugated FITC NPs were also added to activated and inactivated ECs. After internalization, the wells were washed three times with PBS and then lysed by incubating with Triton 1× for 1 hour. An aliquot from each well was collected and analyzed for fluorescence intensity and protein absorption with a microplate reader with excitation and emission wavelength at 430 nm and 485 nm, respectively.

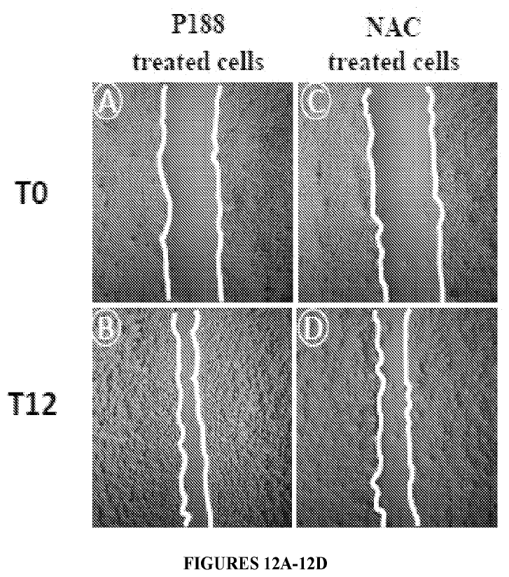

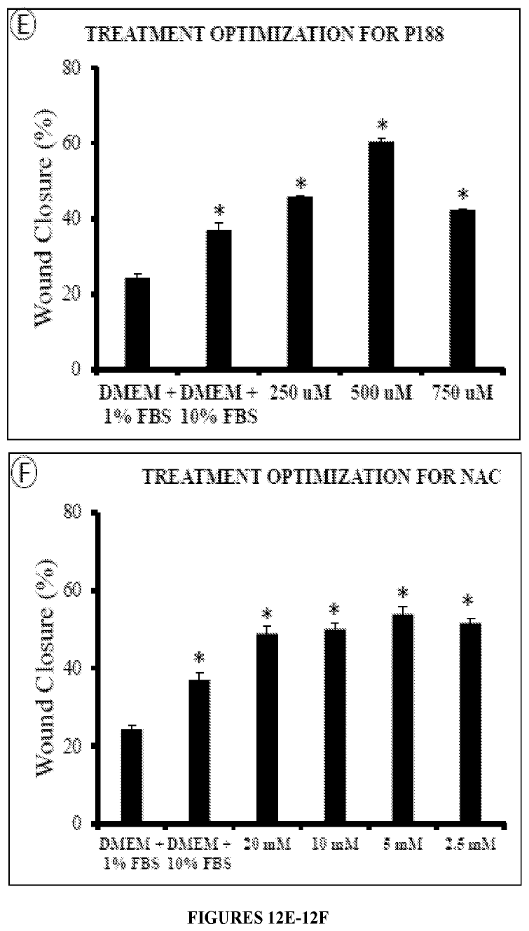

In vitro injury Model and Comparative Assessment of P188 and NAC. Primary mouse endothelial cells were seeded in 24-well plates (10×10³ cells/well) and grown until confluence in complete endothelial cell media. Then a straight scratch was made with a P200 pipette tip to simulate a wound (in vitro scratch wound model). The cell debris was removed by washing with PBS. Wound size images were taken at time, t=0. The wounds were exposed to different single treatments; P188 (500 uM) and NAC (0.82 mg/ml) and in combination (P188+NAC) at the recommended therapeutic concentration in DMEM with 1% serum for 12 hours. The cells without drug were used as the negative control and cells treated with 10% FBS was used as a positive control. Closure of the scratch wound was observed under a microscope (5× magnification) 12 hours after wounding. To quantify the closure of the scratch wound, the difference between the wound width at time 0 and 12 hours was determined. The scratch wound area was measured using ImageJ software (National Institutes of Health, Bethesda, Md., USA). The wound closure efficiency was expressed as a percentage of the wound area normalized by the initial wound area using the equation below, and the results are shown in FIG. 12.

$$S cratch closure rate = \frac{scratch area at time ( h ) 0 - scratch area at time ( h ) 12}{scratch area at time 0} * 100 \qquad ( 2 )$$

Measurement of Endothelial cell permeability. Details of the custom-designed diffusion chamber have been provided elsewhere. FITC Dextran of molecular weight 10 kDa was used. Fluorescent intensity calibration standard was obtained by serial dilution of fluorescent Dextrans. Concentrations in the collected samples were determined using a calibration curve. The permeability coefficient was then calculated using⁶;

$$P = \frac{[ ( C 2 a - C 1 a ) * Va ) ]}{[ A * Δ t * Co ]} \qquad ( 3 )$$

where P is the permeability coefficient, C₂ₐ & C₁ₐ are the concentration in the abluminal chamber at different time intervals, Vₐ is the volume of the abluminal chamber, A is the surface area of the membrane, Δt is the duration of steady-state flux, Co is the concentration in the luminal chamber. The expression (C₂ₐ−C₁ₐ)/Δt is considered as the slope of the diffusion curve over time.

### Example 2: Modulation of In Vitro Brain Endothelium by Mechanical Trauma: Structural and Functional Restoration by Poloxamer 188

ABSTRACT: Brain injuries caused by an explosive blast or blunt force is typically presumed to associate with mechanical trauma to the brain tissue. It has been shown that shockwaves produced by a blast can generate micron-sized bubbles in the tissue. The collapse of microbubbles (i.e., microcavitation) may induce a mechanical trauma and compromise the integrity of the blood-brain endothelium (BBE). To invertigate this, a BBE model was engineered to determine the effect of microbubbles on the structural and functional changes in the BBE. Using monolayers of mouse primary brain microvascular endothelial cells, the permeability coefficient was measured following simulated blast-induced microcavitation. This event down-regulated the expression of tight junction markers, disorganized the cell-cell junction, and increased permeability. Since poloxamers have been shown to rescue damaged cells, the cells were treated with the FDA-approved poloxamer 188 (P188). The results indicate P188 recovered the permeability, restored the tight junctions, and suppressed the expressions of matrix metalloproteinases. The biomimetic interface we developed appears to provide a systematic approach to replicate the structure and function of BBE, determine its alteration in response to traumatic brain injury, and test potential therapeutic treatments to repair the damaged brain endothelium.

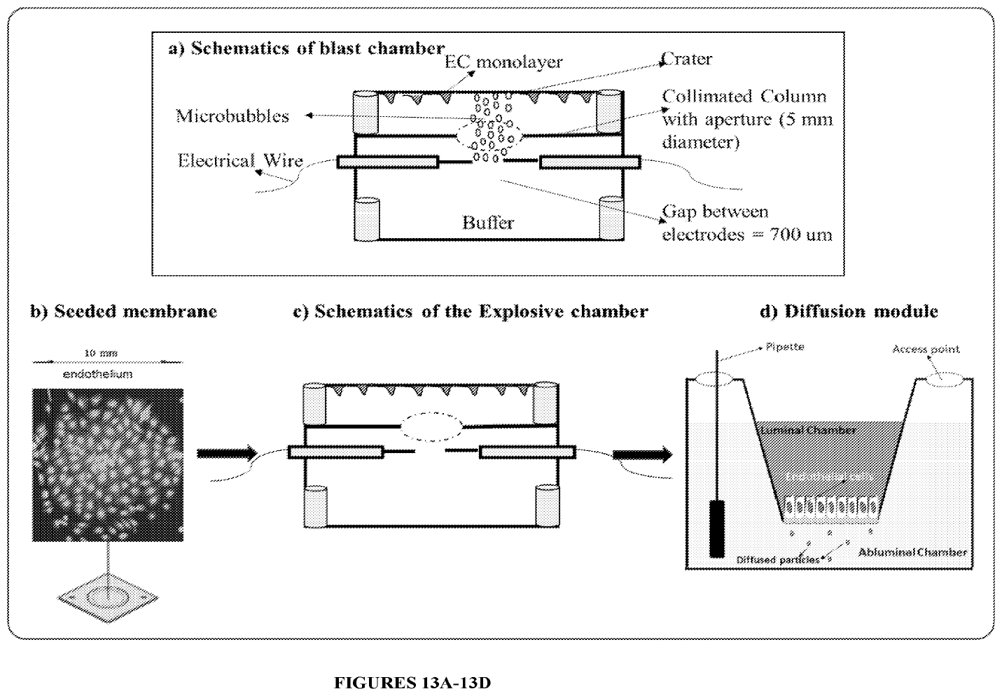

RESULTS: A schematic drawing of the microcavitation/diffusion chamber is shown in FIG. 13. The chamber was used to study the effects of microcavitation. Prior to cell culture, a synthetic polyethylene terephthalate (PETE) membrane (1 um diameter pores) was coated with fibronectin (1 ug/ml). The insert that contains a monolayer of endothelial cells allowed easy handling between the two chambers to expose the cells to microbubbles first (FIG. 13A) and then perform the permeability measurements. To establish the PETE membrane supports cell culture, BECs were pre-incubated with a cell tracker (FIG. 13B) for 30 minutes before seeding on the membrane and shown to reach confluence at day 4. The insert was placed in the microcavitation chamber (FIG. 13C) and then moved to measure the permeability coefficient (FIG. 13D).

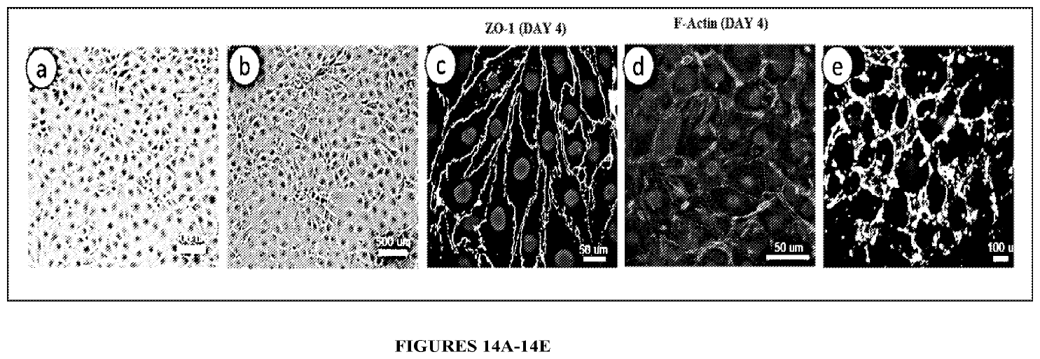

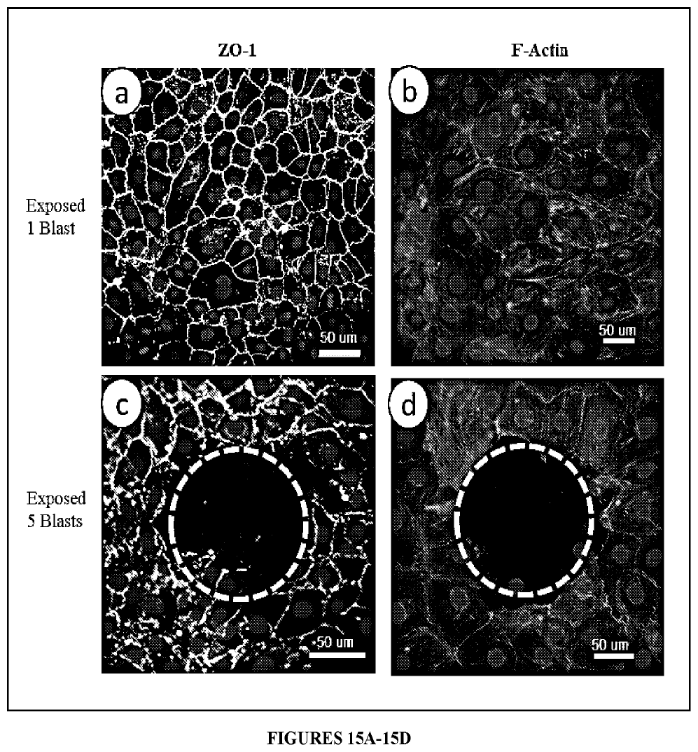

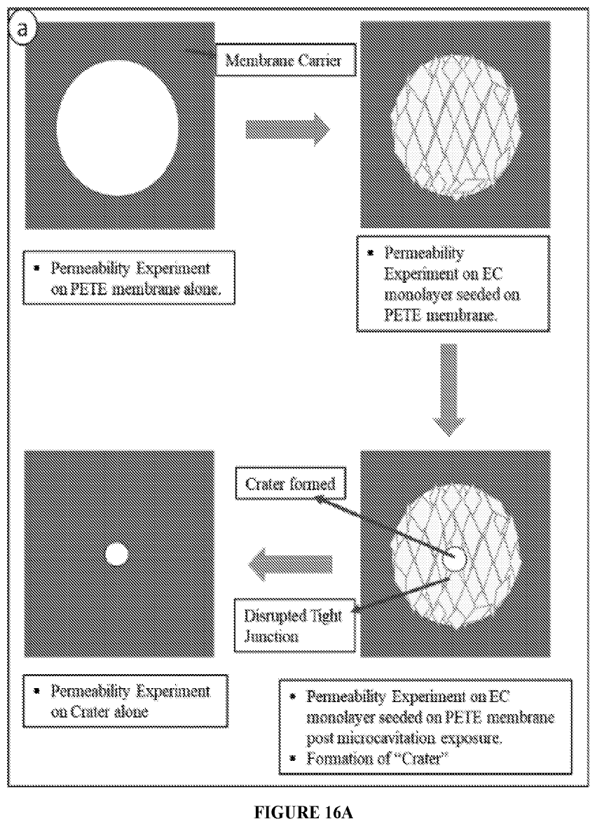

The cells used in this example showed a morphology similar to that of primary cultures of brain endothelial cells and exhibited a monolayer of tightly packed elongated shape that demonstrated cell-cell contact at the confluence (FIG. 14A). At confluence, the cells also showed the spindle-shaped morphology that was previously documented in brain endothelial cells derived from human (FIG. 114B). The cells were also examined for the expression of tight junction protein ZO-1 (FIG. 14C) and F-actin stress fibers (FIG. 14D) at day 4 of culture. The BECs maintained a non-transformed phenotype over more than 6 passages without any sign of senescence. For example, when the cells from passage 5 or 6 were seeded on a reconstituted extracellular matrix (Matrigel), the cells rapidly formed a branched capillary-like cords network, which is characteristic of primary endothelial cells and suggests the normal capacity for angiogenesis (FIG. 14E).

One of the characteristics of physical phenomena repeatedly observed is that the collapse of microbubbles creates a region of interest in which the cells are detached from the substrate. To further illustrate this phenomenon, the endothelial cells were exposed to microcavitation produced by one or five repetitive blasts. In response to a single blast, the cells remained intact, but the tight junctions and F-actins appeared disorganized and disrupted (FIGS. 15A and 15B). In contrast, five repetitive blasts caused cell detachment and created a void as indicated by the dashed line circle (FIGS. 15C and 15D). The region devoid of cells was referred to as a 2D crater. It was estimated that the collapse of microbubble can produce a pressure of ˜55 kPa. These highly pressurized microbubbles can indeed produce a secondary shear stress (e.g., microjet) that is sufficient to detach cells and also impact the cells around the periphery of the crater.

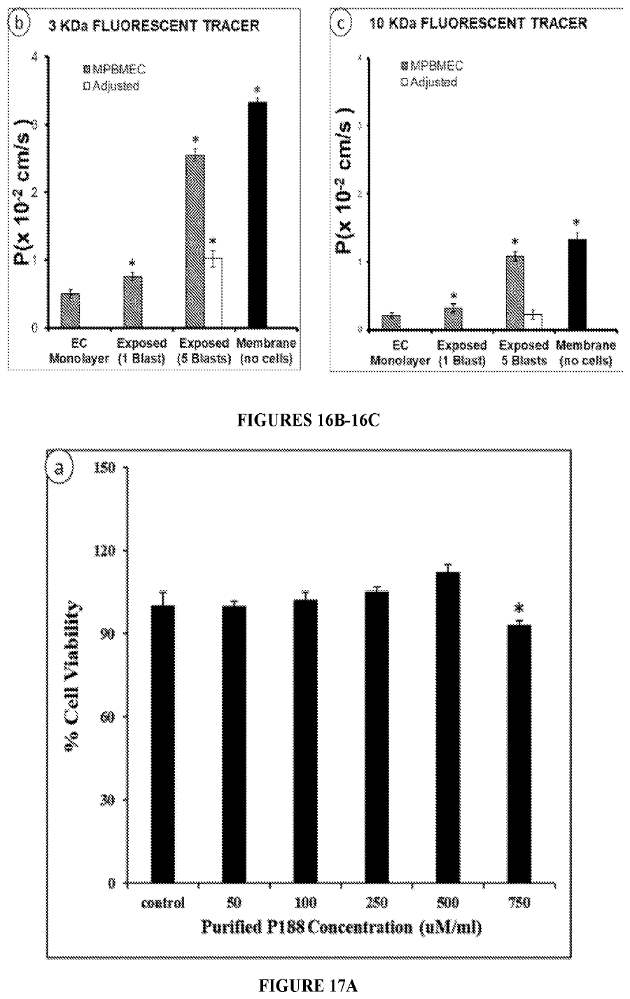

In order to appropriately assess the permeability, diffusion of two tracer molecules was carried out under various experimental conditions. Formation of a crater induced by microcavitation should significantly enhance the permeability and may overwhelm the subtle changes of diffusion through the disorganized tight junctions. Because the location and size of the crater are predictable, a simple microfabrication technique was applied to assess the diffusion through the 2D crater. The experimental procedures are schematically described in FIG. 16A. Using a PETE membrane, we blocked the pores except in an area of a 200 um diameter circle to mimic diffusion through the crater. Adjusted permeability coefficients (P_{adj}) was then calculated by subtracting the permeability coefficient through the crater only without any seeded cells. This adjustment was not needed in response to a single blast (i.e., no crater formed) but was necessary to delineate diffusion through the disorganized tight junctions in response to five repetitive blasts. As expected, the largest permeability coefficient (3.33×10⁻² cm/s) using 3 kDa Dextran was obtained using the PETE membrane alone without cell seeding (FIG. 16B). When a monolayer of endothelial cells was cultured on one side of the PETE membrane, the permeability of 3 kDa Dextran was measured to be more than 6-fold smaller (0.50×10⁻² cm/s). In response to a single blast, the permeability was increased moderately but still statistically significant. However, when five blasts were applied, the unadjusted permeability of 3 kDa Dextran increased noticeably, but P_{adj} was determined to be 2-fold greater than the control value with seeded monolayer of endothelial cells. We performed similar diffusion experiments using 10 kDa Dextran tracers. The permeability coefficients measured were generally much smaller, as expected (FIG. 16C). The permeability of 10 kDa Dextran through the 1 mm pores was about 3 times smaller than that using 3 kDa Dextran. When a monolayer of endothelial cells was cultured, the permeability coefficient of the larger Dextran again decreased from 0.50×10⁻² cm/s (3 kDa) to 0.21×10⁻² cm/s. In response to a single blast, the permeability coefficient remained unchanged (0.23×10⁻² cm/s). Interestingly, a 5-blast exposure did not change the permeability of 10 kDa Dextran when adjusted for diffusion through the crater. The potential implication might be that even though the tight junctions are disrupted, the large 10 kDa molecules are still prevented from diffusing through the disorganized tight junctions.

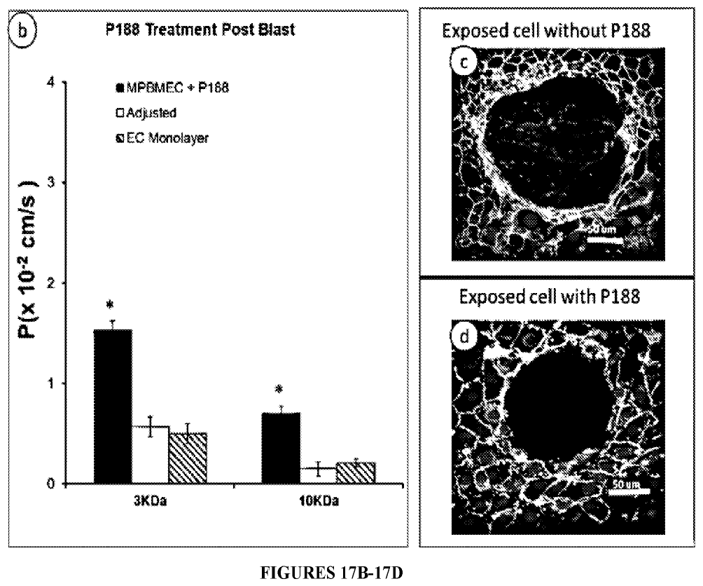

Poloxamer P188 is a surfactant that was originally approved by the FDA as a blood-thinning substance. P188 has since been demonstrated to seal the membrane of damaged cells and repair the cellular function. In the example above, P188 was successfully applied to reconstitute the viability and functionality of astrocytes by restoring the calcium dynamics and minimizing oxidative stress. Prior to applying P188 for potential restoration and repair of the damaged BECs, it was first determined the dose dependence of P188 by varying its concentration from 0 to 750 uM and tested the cell viability using MTT assay (FIG. 17A). After 24 hours, P188 did not induce changes in the cell viability up to the 500 uM concentration. It should be also noted that P188 can form micelle at ˜1 mM concentration⁵¹. Based on these findings, P188 (0.5 mM) was chosen to apply to the injured endothelial cells and potential beneficial effects were determined by measuring the permeability coefficient and examining tight junctions (e.g., ZO-1 gene expression). The Dextran permeability was substantially decreased when the cells were exposed to a 5-blast microcavitation event and then treated with P188 for 6 hrs (FIG. 17B). When adjusted for diffusion through the 2D crater, the reduced permeability was proven statistically significant. The P188 reduced the permeability coefficient of 3 kDa Dextran to 0.57×10⁻² cm/s. This value is essentially indistinguishable from the experimental condition of using a monolayer but without exposure to microbubbles (0.50×10⁻² cm/s). It is interesting to observe that, without treatment of P188, the 2D crater that was created by microcavitation seemed to expand (FIG. 17C) in comparison to when the sample was treated with P188 (0.5 mM) for 6 hrs (FIG. 17D). Taken collectively, the P188 treatment appears to restore the biotransport properties and stabilize the damaged area (i.e, crater).

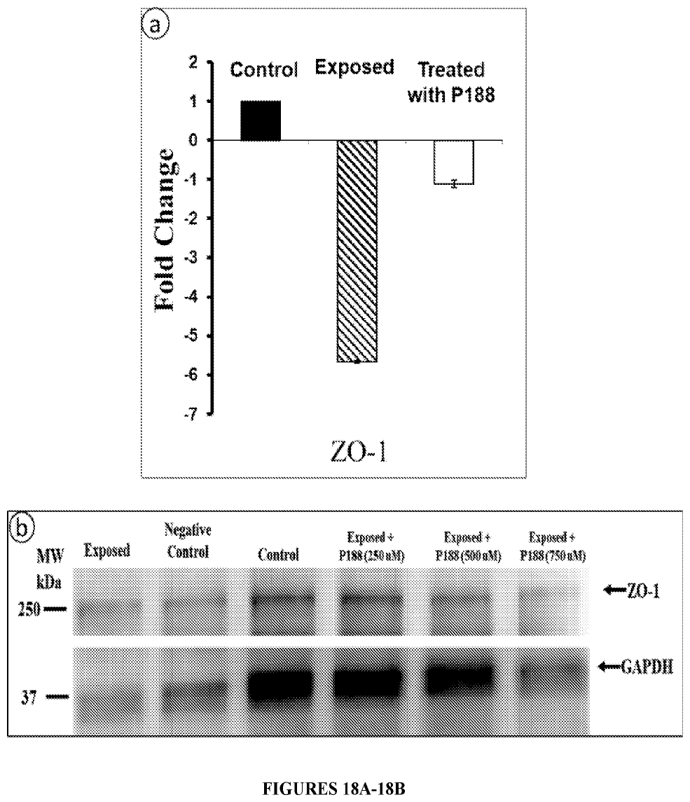

The effect of microcavitation on tight junction was further probed by determining the ZO-1 gene expression. A 6-fold decrease was measured. In contrast, when P188 was applied for 6 hours following a microcavitation event, the ZO-1 expression was significantly restored (FIG. 18A). In addition to PCR experiments, we performed Western blot analysis for ZO-1 using BECs. The ZO-1 protein level was significantly down-regulated in response to microcavitation (FIG. 18B). Consistent with the gene expression results, endothelial cells treated with P188 showed at least a partial restoration of the protein level. Furthermore, since the protein level was measured in no serum media when the cells were exposed to microcavitation, BECs were incubated in no serum media (e.g., negative control). The results indicate that the media condition was not responsible for the P188-induced restoration of the ZO-1 protein level.

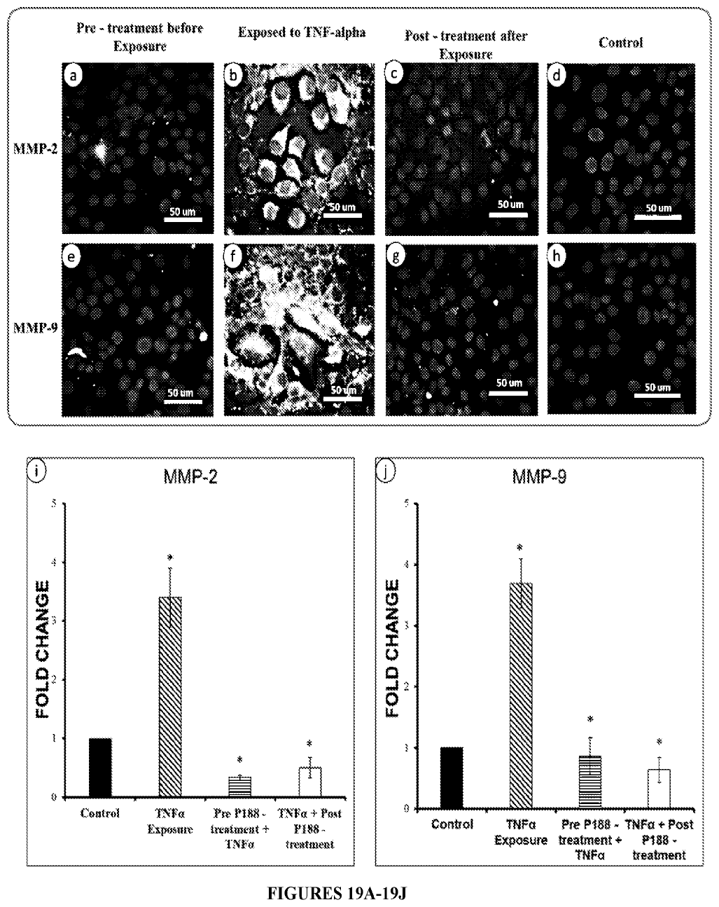

To probe the correlation between ZO-1 and matrix metalloproteinases (MMPs), specifically MMP-2 and MMP-9, a chemical trauma (TNF-α) was first introduced to endothelial cells. As shown in (FIGS. 19D & 19H), there was essentially no expression of MMP-2 or MMP-9 in control cells. Treatment of cells with TNF-α induced measurable changes and upregulated both MMP-2 and MMP-9 (FIGS. 19B & 19F). More interestingly, cells were found to down-regulate the MMPs if they were treated with P188 either before (FIGS. 19A and 19E) or after (FIGS. 19C and 19G) exposure to TNF-a. Fluorescent images indicate P188 indeed appears to effectively regulate the MMP expressions. To quantify the effect of P188 on the MMP expressions, PCR experiments were performed to determine the gene expression of MMP-2 and MMP-9 (FIGS. 191 and 19J). Approximately a 4-fold increase in the MMP-2 and MMP-9 gene expressions were induced in response to TNF-α. Either pre- or post-treatment of cells with P188 mitigated the effect of inflammatory trauma and down-regulated the MMP expressions.

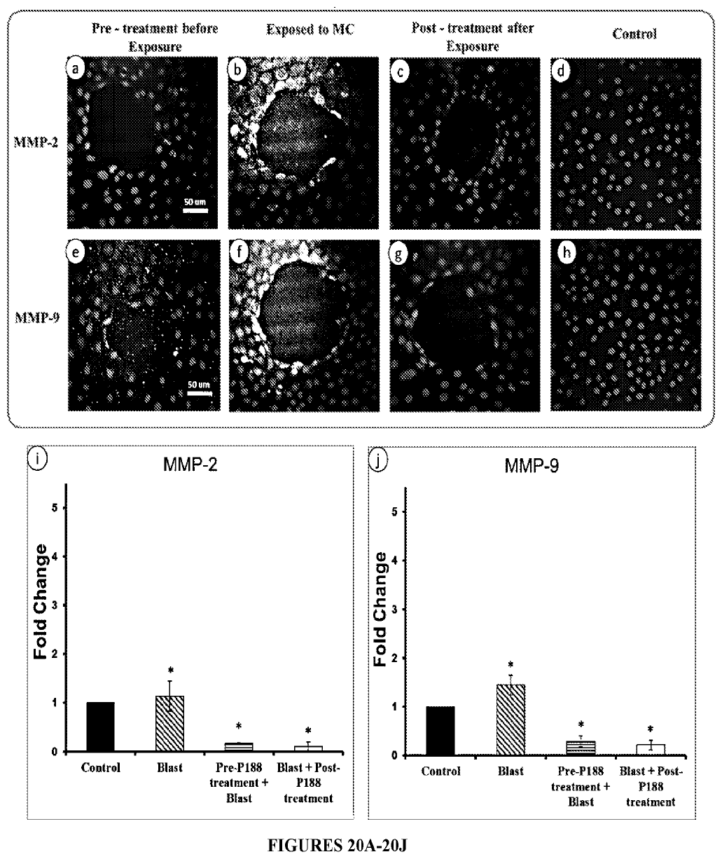

Whether P188 can also minimize the MMP expressions in response to a mechanical trauma such as microcavitation was determined. As illustrated in FIG. 20, similar results that the MMP expressions were up-regulated by microcavitation was observed. However, the fluorescent image intensities would suggest the extent of the MMP upregulation was not as extensive as that induced by TNF-α. Nonetheless, treating the cells with P188 either prior to or following a microcavitation event was effective in significantly down-regulating the MMP expressions (FIG. 20A to 20H). Quantitative measurements of the MMP expressions are also consistent with the fluorescent images. However, unlike the 4-fold increase in the MMP gene expressions induced by TNF-α, the microcavitation induced about a 50% increase (FIGS. 20I and 20J).

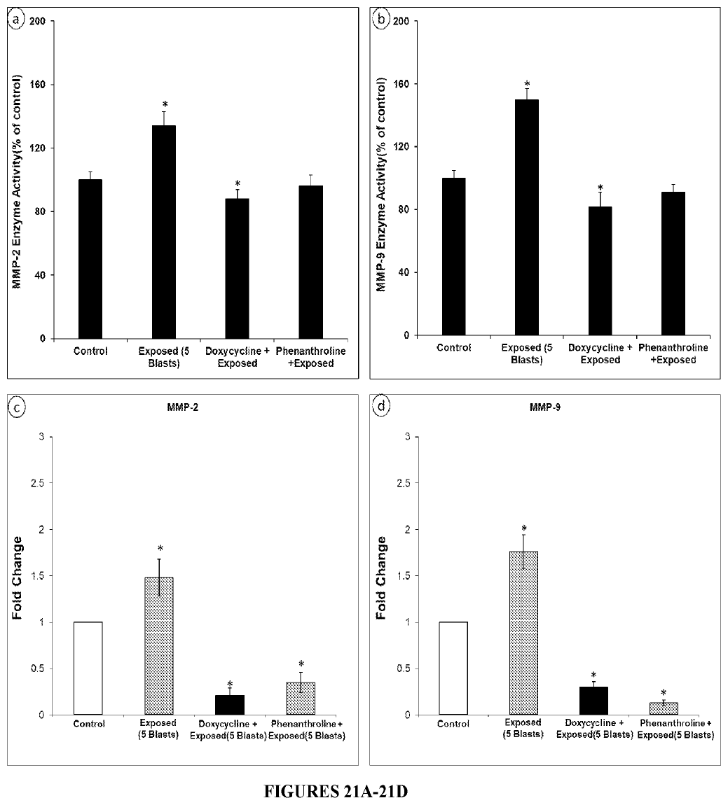

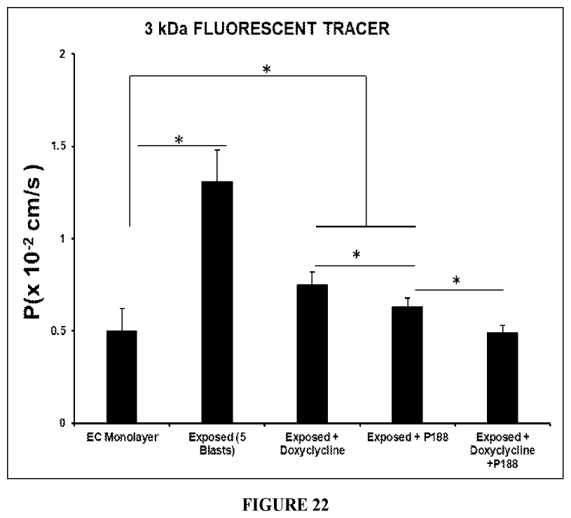

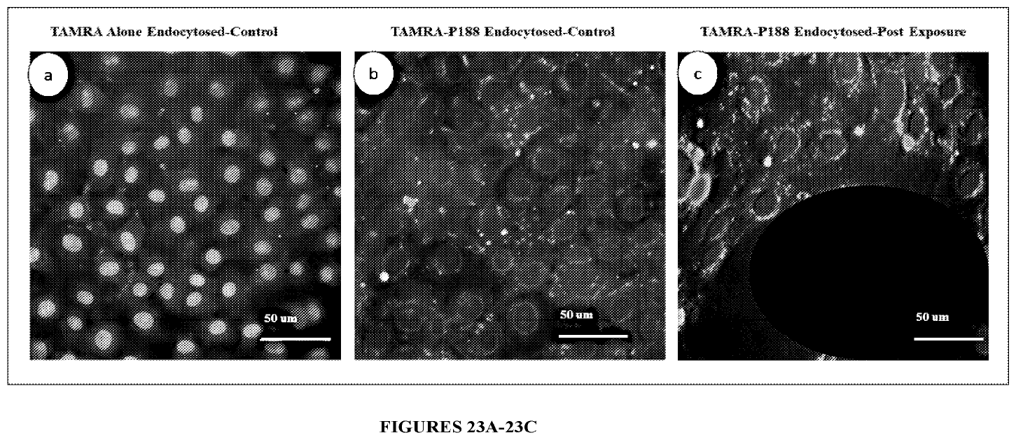

To examine whether P188 directly inhibits MMP-2 & 9, the MMP-2 & 9 activities and gene expressions were measured after treating the cells with doxycycline. Briefly, primary brain endothelial cells were grown under different conditions-control (no treatment), exposed to microcavitation, treated with doxycycline (20 mM) and exposed to microcavitation, and treated with phenanthroline (0.5 mM; membrane permeable metalloprotease inhibitor). Endothelial cells exposed to microcavitation significantly increased the MMP-2 & 9 enzymatic activity when compared to control. The increased activity was negated with doxycycline pretreatment (FIGS. 21A and 21B). To further validate the results, the gene expressions were also quantified to demonstrate the decreased MMP activities were consistent with down-regulation of the two genes (FIGS. 21C and 21D). Primary brain endothelial cell monolayers exposed to microcavitation displayed a significant increase in the permeability of tracer molecules. Such an increase in permeability is expected to be mitigated when the MMP activates and gene expressions were modulated by doxycycline. Following a microcavitation event, doxycycline was shown to reduce the permeability. However, it was not as efficient as treating the cells with P188 (FIG. 22). Interestingly, the combination of doxycycline and P188 together was observed to completely restore the permeability to the control level, suggesting that P188 is likely to inhibit intracellular signaling pathways other than those affected by doxycycline.

DISCUSSION: Traumatic brain injury can be induced by blunt force to the head, chemical factors and also shockwaves from explosive blasts. The formation of micron-size bubbles and the subsequent collapse of the microbubbles have been described. The term microcavitation was coined to describe the process and was able to quantitatively study the interaction between brain endothelial cells and the collapse of highly pressurized microbubbles. The collapse of microbubbles (˜55 kPa; ˜10 s interaction time) was observed to detach cells from the substrate and created a region that is devoid of cells. The area of cell detachment has been referred to as a crater. Examples of the crater have been shown in FIGS. 15 and 17. Because TBI typically leads to activation of inflammatory responses and release of free radicals which, in turn, could cause necrosis and eventual apoptosis, tissue engineering approaches were used to develop a model to examine the altered biotransport properties of the blood-brain endothelium and identified key molecules that contributed to dysfunctional endothelium.

There are several models of blood-brain barrier disruption but very few, if any, focuses on the mechanism(s) responsible for blood-brain endothelium disruption. The model allows measurement of the permeability that might have been modulated due to microcavitation. Using two different molecular weight Dextran, the permeability coefficients under various experimental conditions were measured (see FIG. 16). It was recognized that, since microcavitation created a region of detached cells, diffusion of Dextran molecules should be primarily through the crater. It is difficult then to delineate the measured permeability coefficients and determine the contribution to the increased permeability that is associated with disorganized tight junctions. This challenge was resolved by a unique engineering design in which a substrate was physically blocked, except the molecules were allowed to diffuse through the crater. The measured permeability coefficients were then adjusted for the unhindered diffusion through the crater. Such an adjustment reduced the permeability of the smaller 3 kDa Dextran molecules by almost ˜3-fold (FIG. 16). However, the adjusted permeability coefficient was nonetheless significantly greater than the permeability through unexposed blood-brain endothelium. When the same experiments were carried out using 10 kDa Dextran, the adjusted permeability coefficient did not differ from the control value. One plausible interpretation would be that even though we observed tight junction disruption, larger molecules are still excluded from diffusing through the disorganized tight junctions. In contrast, diffusion of the smaller molecules <3 kDa appears to be enhanced through tight junctions. Since toxins in the blood are typically small molecules, they can more easily cross the brain endothelium through the disrupted tight junctions. Eventually, it leads to thickening of the capillary basement membrane, degeneration of small cerebral arteries, and defective regulation of cerebral blood flow.

Traumatic brain injury causes activation of inflammatory responses and release of free radicals, which leads to necrosis and eventual apoptosis. In response to either chemical (TNF-α) or mechanical (microcavitation) trauma, the MMP expressions are significantly up-regulated. Moreover, the tight junction formation and function appear to inversely correlate with the MMP expressions. Interestingly, our results indicate treating the disrupted endothelial cells for 3 hours with P188 promoted tight junction recovery by down-regulating MMP-2 & 9 and up-regulating ZO-1. Results from the present examples support the therapeutic effects of P188. For example, the tight junction protein (ZO-1) in BECs exposed to microcavitation was suppressed, while a treatment of the injured cells with P188 showed upregulation of the tight junction protein (FIG. 18). The beneficial effects of P188 are reproducible and statistically significant. A few studies suggest that P188 might inhibit the NF-kB signaling pathway. If validated, it would provide a clearer understanding of the interaction between P188 and NF-kB that is a known transcription factor for MMP expressions. In an animal model study, it has been demonstrated that P188 exerts neuroprotective effects by preventing MMP-mediated tight junction degradation. In a stroke animal model study, P188 has again been shown to provide protection against cerebral ischemia through inhibition of MMP-9.

Maintenance of the BECs relies on the interaction of endothelial cells and basement membrane. Together, they play an essential role in the biotransport of molecules from the blood to the brain and vice versa. TBI is typically accompanied by an increased permeability of the BBB. Studies have shown that an increased level of MMP-2 & -9 plays a key role in the BBB destruction by induction of inflammatory cascade reaction. The activity of the two proteolytic enzymes is associated with the BBB permeability and facilitates degradation of tight junction proteins. Conversely, inhibition of MMP-2 & -9 promotes recovery of the BBB. Therefore, targeting MMP-2 & -9 may be one of the strategies for the treatment of TBI. Our results show that the excessive expression of MMP-2 & -9 can be reversed by P188 treatment in response to an inflammatory (FIG. 18) or mechanical trauma (FIG. 19). As a confirmatory study, doxycycline was applied to diminish the MMPs. Microcavitation-induced upregulation of both the MMP activities and gene expressions were shown to be mitigated by treating the cells with doxycycline (FIG. 21). The effect of doxycycline was used to examine the permeability through tight junctions (FIG. 22). As expected, doxycycline not only inhibited the upregulation of MMPs but also significantly restored the permeability. It should be mentioned that when the combination of doxycycline and P188 was applied together, the altered permeability that was attributed to microcavitation was restored to the control value. These in vitro data provide additional evidence that P188 along with an adjuvant therapeutic compound could offer a viable treatment in the TBI patients.

As a first step toward a better elucidation into how P188 interferes with downregulation of MMPs, P188 was conjugated with a fluorescent dye (TAMRA) and incubated BECs with the conjugated P188. As shown in FIG. 23, the TAMRA dye alone was able to diffuse through the cell membrane and, in some cells, it appears to have penetrated into the nucleus (FIG. 23A). When the TAMRA-P188 complex was introduced to BECs, P188 was shown to be internalized in control cells but excluded from the nucleus (FIG. 23B). In response to microcavitation, the cells on the periphery of the crater show an increased fluorescence intensity. It may be likely due to an influx of the complexes through the compromised cell membrane. Further, it was observed in some cells that the P188-TAMRA complexes penetrated inside the nuclei of the injured cells, suggesting compromised nuclear transport function. Although more systematic studies are indeed required to reveal the molecular interactions that involve P188 in the injured cells, it appears plausible that P188 can be coupled to the intracellular signaling pathways as well as in yet to be defined nuclear interactions.

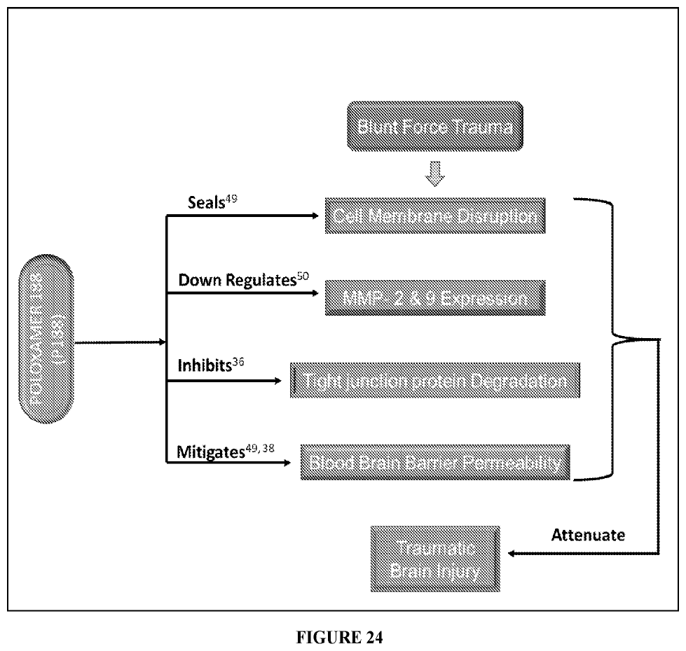

CONCLUSION: In summary, established and characterized were an in vitro brain endothelium model using a biopolymeric membrane. Using the model, changes in the biotransport properties were experimentally quantified in response to microcavitation and the potential restorative effects of P188. The role of P188 to repair the injured endothelium in response to an inflammatory or mechanical trauma was also established. Although the specific molecular mechanism(s) mediating the therapeutic effects of P188 are under investigation, such effects are likely found at the level of the cell membrane, cytoplasm, between cells, and even in the nucleus. Finally, the model developed and used in this example is quite simple, easy to engineer, convenient, and reproducible to provide a platform for studies of molecular mechanisms and rapid screening for potential therapeutic compounds. Based on collective findings from the study, a working model was postulated and proposed that P188 delivers therapeutic effects at multiple levels (FIG. 24). P188 seals the disrupted cell membrane, downregulates MMPs, and subsequently restores the structural and functional integrity of tight junctions. This leads to restore and repair the biotransport properties of the brain endothelium. These multi-level beneficial effects of P188 are expected to attenuate the extent of traumatic brain injury.

Materials and Methods

In vitro Brain Endothelium Model: Mouse brain endothelial cells were cultured on a synthetic polyethylene terephthalate (PETE) membrane (Sterlitech, Kent, Wash.) that contains 1 μm diameter pores at the density of 2×10⁷ pores/cm². This well-defined membrane allowed us to establish a monolayer of endothelial cells and determine the endothelium permeability. Balb/c mouse primary brain microvascular endothelial cells (MPBMECs; Cell Biologics Inc., Chicago, Ill.) were grown in endothelial basal medium-2 (EBM-2) with EGM-2 kit (Lonza, Walkersville, Md.) in a flask coated with a gelatin-based coating solution. Cells were seeded at a density of 6.60×10⁴ cells/cm² on the PETE membrane coated with fibronectin (1 μg/ml). To confirm the viability of MPBMECs, an in vitro angiogenesis was performed. Briefly, reduced growth factor Matrigel (200 uL; Corning, Corning, N.Y.) was pipetted onto a 22×22 mm coverslip in a petri dish and incubated at 37° C. and 5% CO₂ for 30 min to solidify Matrigel. Once Matrigel was set, approximately 5×10⁴ cells MPBMECs were seeded on the gel and tube formation was observed after 6 hrs. To accurately visualize the tube formation, FITC cell tracker was added to the cells before seeding.

Microbubble Exposure Chamber: A description of the simulated blast and microbubble chamber used in this experiment is known. This chamber is designed to fit the microscope stage for real-time imaging. Briefly, two platinum electrodes were symmetrically embedded in the middle of the chamber (˜10 mm high; FIG. 13A). Shockwaves were generated by applying an electrical pulse across the electrodes that were separated by 700 um. Temporary electrical breakdown of water creates shock waves, followed by the generation of microbubbles. Potential artifacts such as temperature rise, excessive-high electric field, and the impact of shock waves were all carefully assessed, measured, and addressed previously. For example, a temperature rise of <0.5° C. was measured²¹, and the peak shock wave pressure was recorded 10 MPa. The pressure of collapsing microbubbles was also measured to be 55 kPa, which is at least two orders of magnitude smaller than the peak pressure but certainly large enough to adversely impact the cells at or near the site of microcavitation. The principle of symmetry was applied to study the effect of shock waves by plating cells at the bottom of the chamber (FIG. 13A). Should the shock waves (0.3 us travel time across the depth of the chamber) cause any measurable impact, these effects should be observed both at the top and bottom of the chamber. The cells plated at the bottom of the chamber were observed viable and functional and expressed, for example, the baseline fluorescent markers of apoptosis. In contrast, the microbubbles rose only to the top of the chamber and collapsed onto the plated cells. The density of microbubbles, their speed, and size distribution following propagation of shock waves were recorded, analyzed, and reported. The cellular and molecular signatures that are associated with endothelial cell damage are yet to be determined.

Immunostaining for ZO-1, Actin, and MMP-2 & 9: Tight junctions in the brain endothelial cells were visualized using zonula occludens 1 (ZO-1) monoclonal antibody conjugated with Alexa Fluor 488 (Thermofisher Scientific, Waltham, Mass.). The BECs were fixed with 4% paraformaldehyde in PBS for 10 minutes at room temperature and blocked with 3% of BSA for 1 hour. Cells were incubated with fluorescently conjugated ZO-1 monoclonal antibody for 1 hour and kept in the dark. Nuclei were stained with DAPI at a dilution of (1:1000). For MMP-2 & 9 analyses, cells were fixed with 4% paraformaldehyde in PBS, permeabilized with 0.1% Triton X-100 for 3 minutes and blocked with 3% of BSA for 1 hour. Cells were incubated with the MMP-2 & 9 primary antibodies (1:500, 4.3 μg/ml, Santa Cruz Biotechnology, Santa Cruz, Calif.) and subsequently incubated with Alexa Fluor 488-conjugated goat anti-rabbit IgG (1:1000, 2 μg/ml, Santa Cruz Biotechnology).

RNA Isolation and Gene Expression Measurements: Total RNA was isolated according to protocols supplied by the manufacturer (Quick-RNA Microprep Kit, Zymogen Research Corp, Irvine, Calif.). Briefly, cells were lysed in an RNA lysis buffer washed with 95% ethanol and RNA prep buffer, and then sequentially washed with RNA wash buffer, and RNA was eluted with 15 ul of DNase/RNase-Free water. The sample was treated with RQ1 RNase-Free DNase from (Promega Life Sciences, Madison, Wis.) to remove any remaining DNA. NanoDrop Spectrophotometer was used to measure the 260/280 ratio as well as RNA yield (quantity, ng/ul). RT-PCR was carried out using the manufacturer's protocols. cDNA was synthesized using AzuraQuant cDNA Synthesis kit (Azura Genomics, Raynham, Mass.). The mastermix was prepared using the Green Fast qPCR Mix Hirox Kit (Azura Genomics). Two microliters of cDNA were used in each subsequent PCR reaction. Specific primers for BBB tight junction protein; zonula occludens 1 (ZO-1), and a housekeeping gene, GAPDH (RealTimPprimers.com), were designed and synthesized according to published sequences (RealTimePrimers.com). PCR amplifications were performed in a final volume of 20 ml of the mastermix using AzuraQuant Green Fast qPCR Mix Hirox as instructed by the manufacturer. Using applied biosystems 7300 Fast Real-Time PCR system (Thermo Fisher Scientific), amplifications were done for 45 cycles using a denaturation step at 95° C. for 5 s, an annealing step at 60° C. for 25 s, and polymerization step at 72° C. for 45 s.

Western blots: Endothelial cells were washed twice with PBS and lysed with RIPA buffer (Sigma) plus protease inhibitor cocktail (Sigma). Protein concentration was quantified via Coomassie Protein Assay Reagent (Thermos Scientific), and proteins were then resolved by Mini-PROTEAN TGX Stain-Free Gel (BIO-RAD) before transferring to 0.2 um PVDF membrane in Trans-BLOT Turbo Transfer Pack (BIO-RAD) using the Trans-Blot Turbo Transfer Stater System Mini. Blocking was conducted for 1: 30 h in Tris-buffered saline (10 mM Tris-HCl, 100 mM NaCl, pH7.5) containing 0.1% Tween-20 (TBST) and 5% BSA. Samples were probed overnight at 4° C. with rabbit polyclonal ZO-1 tight junction protein antibody (1:2000, Abcam) and GAPDHS rabbit polyclonal antibody (1:1000, Proteintech) diluted in TBST with 5% BSA. After being washed three times for five minutes with TBST, the sample was incubated with goat anti-rabbit IgG H&L (HRP) (Abcam) for 1 h at room temperature. The membrane was washed three times for five minutes with TBST before developing/enhancing the signal using Clarity Max Western ECL Substrate. The image was acquired using ChemiDoc Touch MP Imaging System and analyzed with Image Lab Software.

MMPs activities assay, Fold change, and Permeability measurement: EnzChek MMP-2 & 9 Assay Kit were used for MMP-2 & 9 activity assays. Cells were cultured in endothelial cell complete media and divided into the following groups: control (no treatment), exposed (5 blasts), doxycycline (20 mM)+exposed (5 blasts), doxycycline (20 mM), and phenanthroline (0.5 mM, positive control). Before initiating experiments, cells were washed with PBS, and no serum media was applied. Dishes in the treatment groups were pretreated with doxycycline or phenanthroline for 2 h at 37° C. Cells were exposed to microcavitation and incubated at 37° C. for 1 h. All groups were homogenized with lysis buffer containing 0.1% Triton-X 100, and protein was estimated with Pierce BCA Protein Assay Kit. Equal amounts of protein from each sample were aliquoted in lysis buffer. Samples were then analyzed using a plate reader at excitation 495 nm and 515 nm to quantitate MMP-2 & 9 activities. Primary brain endothelial cells were grown under the conditions described above. At the end of all experimental conditions, RNA from the cells was extracted and used for PCR analysis of the MMP-2 & 9 gene expressions. Finally, the permeability of the brain endothelium model exposed to doxycycline, P188, or in combination of them both was measured.

In Vitro Diffusion Model and Permeability Experiment: The permeability experiments were carried in a 2-step process (FIG. 13B-13D). After seeding and culturing cells on a portable insert, it was transferred to the microbubble exposure chamber (see above). Following a microcavitation event, the insert was then placed in a custom-designed diffusion chamber. The insert that contains the endothelial cells was mounted into the chamber, and its edge was sealed to prevent leakage. With the insert properly secured in the upper chamber, it was then filled with PBS containing FITC Dextran molecules. The lower chamber was filled with 5 ml of PBS without phenol red. The permeability experiment was conducted in the dark and at room temperature. FITC Dextran of molecular weight 3 or 10 kDa were used for the experiment. A small volume of 25 μl was collected from the lower chamber at one-hour intervals for 6 hours and stored at −80° C. Fluorescence intensities were measured using a fluorescence plate reader (Synergy HT). Fluorescent intensity calibration standard was obtained by serial dilution of fluorescent Dextran. Concentrations in the collected samples were determined from the calibration curve. The Permeability coefficient was then calculated by modifying the equation from Li et al.,²⁹;

$$P = \frac{[ ( C 2 a - C 1 a ) * Va ) ]}{[ A * Δ t * Co ]} ,$$

where P is the permeability coefficient, C₂ₐ & C₁ₐ are the concentration in the abluminal chamber at different time intervals, Vₐ is volume of the abluminal chamber, A is the surface area of the membrane, Δt is the duration of steady-state flux, Cₒ is the concentration in the luminal chamber. The expression (C₂ₐ−C₁ₐ)/Δt is considered as the slope of the diffusion curve over time.

Dose-dependent study of P188: Dose-dependent study on the proliferation of primary brain endothelial cells was measured using CellTiter 96® AQueous One Solution Cell Proliferation Assay for 24 and 48 hours. It is a colorimetric method for determining the number of viable cells in proliferation or cytotoxicity assays. It is non-toxic, which allows an extended period of incubation. Briefly, cells were incubated with CellTiter 96® AQueous One Solution Cell Proliferation Assay for an hour. Absorbance was recorded at 490 nm using a plate reader, and the plate returned to an incubator until 48-hour time point. Untreated cells were used as control. The viability of cells following a 24-hour exposure to P188 was assessed with the MTT assay. Briefly, after exposure of BECs to P188, cells were treated with MTT (5 mg/ml) for 1 hour at 37° C. The formazan product generated was solubilized by addition of 20% sodium dodecyl sulfate and 50% N, N-dimethylformamide, and quantified by measuring its absorbance at 490 nm. Resulting sample absorbance was used to calculate cell viability.

Statistics: All experiments were replicated at least three times. Data are presented as the mean±standard deviation (SD) and analyzed using Student's t-test or ANOVA with a posthoc test. p<0.05 was indicated with an asterisk (*).

1. A composition comprising:
a population of polyester derived nanoparticles; wherein each polyester derived nanoparticle comprises:
a) a therapeutic agent encapsulated therein for treating traumatically injured, inflamed, diseased, or disrupted endothelial cells, and
b) a targeting ligand bound to the nanoparticle, wherein the targeting ligand binds to a biomarker for the injured, inflamed, diseased, or disrupted endothelial cells.

2. The composition of claim 1, wherein the population of nanoparticles has an average particle size ranging from about 10 nm to about 1000 nm, such as from about 100 nm to about 500 nm, or from about 120 nm to about 300 nm.

3. The composition of claim 1, wherein the population of nanoparticles has a polydispersity of 0.1 or less.

4. The composition of claim 1, wherein the polyester derived nanoparticles comprises polylactic acid (PLA), poly glycolic acid, poly (lactic-co-glycolic acid; PLGA) or combinations thereof.

5. The composition of claim 4, wherein the polyester derived nanoparticle comprises poly (lactic-co-glycolic acid) having a weight average molecular weight of 5,000 to 100,000; from 10,000 to 50,000; or from 24,000 to 30,000.

6. The composition of claim 1, wherein the therapeutic agent when administered alone, exhibits negligible to no oral bioavailability.

7. The composition of claim 1, wherein the therapeutic agent comprises a non-biologic material.

8. The composition of claim 1, wherein the therapeutic agent comprises a surfactant.

9. The composition of claim 1, wherein the therapeutic agent comprises a poloxamer surfactant, such as poloxamer 188 (P188).

10. The composition of claim 1, wherein the therapeutic agent further comprises an antioxidant, doxycycline, or a combination thereof.

11. The composition of claim 9, wherein the antioxidant comprises N-acetylcysteine (NAC).

12. The composition of claim 1, wherein the composition further comprises an additional therapeutic agent.

13. The composition of claim 1, wherein the therapeutic agent and nanoparticles are present in a weight ratio from 50:1 to 500:1, from 100:1 to 500:1, or from 150:1 to 400:1.

14. The composition of claim 1, wherein the targeting ligand is selected from an antibody, a small molecule, a peptide, a carbohydrate, an siRNA, a protein, a nucleic acid, an aptamer, a second nanoparticle, a cytokine, a chemokine, a lymphokine, a receptor, a lipid, a lectin, a ferrous metal, a magnetic particle, a linker, an isotope and combinations thereof.

15. The composition of claim 1, wherein the biomarker is for traumatic injury, preferably traumatic brain injury.

16. The composition of claim 1, wherein the biomarker is selected from P-selectin, E-selectin, or L-selectin.

17. The composition of claim 1, wherein the targeting ligand comprises P-selectin glycoprotein ligand-1 (PSGL-1).

18. A method of treating traumatic injury in a subject in need thereof comprising, administering to the subject a therapeutically effective amount of the composition of claim 1.

19. The method of claim 18, wherein the composition exhibits a burst release followed by a sustained release of one or more of the therapeutic agent.

20. The method of claim 18, wherein the therapeutic agent comprises a surfactant for repair of endothelial cells and an antioxidant.

21. The method of claim 20, wherein the composition exhibits an immediate burst release followed by a sustained release of the surfactant; and an immediate burst release of the antioxidant.

22. The method of claim 18, wherein the composition is administered intranasally, orally, parenterally, subcutaneously, pulmonarily, intravenously, intraperitoneally, intrathecally, intracerbrally, epidurally, intramuscularly, intradermally, or intracarotidly.

23. The method of claim 18, wherein the composition is administered intranasally or orally.

24. The method of claim 18, wherein the subject is diagnosed with an acute traumatic brain injury or a chronic traumatic brain injury.

25. The method of claim 18, wherein the method repairs leaky endothelium.

26-32. (canceled)
